(19) United States
(12) Patent Application Publication
(10) Pub. No.: US 2017/0253658 A1
(43) Pub. Date: Sep. 7, 2017

(54) CD94/NKG2A AND/OR CD94/NKG2B ANTIBODY, VACCINE COMBINATIONS

(71) Applicant: ACADEMISCH ZIEKENHUIS LEIDEN H.O.D.N. LUMC, LEIDEN (NL)

(72) Inventors: SJOERD VAN DER BURG, LEIDEN (NL); THORBALD VAN HALL, LEIDEN (NL)

(21) Appl. No.: 15/506,263

(22) PCT Filed: Aug. 28, 2015
(86) PCT No.: PCT/NL2015/050600

§ 371 Date: Feb. 24, 2017

(30) Foreign Application Priority Data
Aug. 28, 2014 (EP) 14182708.9

(51) Int. Cl. C07K 16/28; A61K 39/395; A61K 35/17; A61K 39/00

(52) U.S. Cl. CPC C07K 16/2851; C07K 16/2803; A61K 39/0011; A61K 39/39558; A61K 35/17; C07K 2317/92; C07K 2317/24; A61K 2039/507

(57) ABSTRACT

The disclosure provides among others a combination of a vaccine and a CD94/NKG2A and/or a CD94/NKG2B binding antibody for use in the treatment of a subject in need thereof, wherein said vaccine comprises an immunogen for eliciting an immune response against an antigen or a nucleic acid molecule encoding said immunogen.

The invention relates to the field of immunotherapy. The invention in particular relates to CD94/NKG2A/B antagonists, preferably antagonistic CD94/NKG2A/B antibodies in combination with vaccines or immunogens to stimulate an immune response. The invention is particularly but not exclusively useful in the treatment of cancer.

Immune checkpoint blocking antibodies to CTLA-4 and PD-1 on tumor-infiltrating T cells have resulted in significant clinical responses in late stage cancer patients. CTLA-4 is expressed on several T-cell subsets and activated cells, as witness of a negative feedback loop. Anti-CTLA-4 antibodies represent an example for a first-in-class therapeutic. Clinical trials with anti-PD1 and anti-PD-L1 antibodies also show clinical results.

In the present invention we observed that activated CD8 T cells (CTL) and natural killer (NK) cells express the inhibitory receptor CD94/NKG2A. Its ligand is the conserved HLA-E molecule. A unique feature of CD94/NKG2A is that it is a negative regulator on CTL and NK cells, both involved in direct tumor control. We further observed that HLA-E expression by tumors correlates with a poor survival in CD8 cell infiltrated tumors otherwise showing good survival.

In the experimental section we provide among others evidence that CD94/NKG2A-blockade allows a good response by intratumoral CTL and NK cells to tumors. VIN patients with high NKG2A-positive CTL numbers have a better progression-free survival. Up to 50% of tumor infiltrating CTL of head&neck cancers, ovarian cancers and cervical cancers express NKG2A. Around 30% of these NKG2A-positive CTL do not express other co-inhibitory receptors TIM3, CTLA-4 or PD-1. The frequency of NKG2A-positive CTL in the tumor increase upon therapeutic vaccination. The expression level of an NKG2A ligand on tumor cells is increased upon therapeutic vaccination.

### SUMMARY OF THE INVENTION

The invention provides a combination of a vaccine and a CD94/NKG2A and/or a CD94/NKG2B binding antibody or a CD94/NKG2A and/or a CD94/NKG2B binding part thereof for use in the treatment of a subject in need thereof, wherein said vaccine comprises an immunogen for eliciting an immune response against an antigen or a nucleic acid molecule encoding said immunogen.

The invention further provides a pharmaceutical composition comprising vaccine and a CD94/NKG2A and/or a CD94/NKG2B binding antibody or a CD94/NKG2A and/or a CD94/NKG2B binding part thereof, wherein said vaccine comprises an immunogen for eliciting an immune response against an antigen or a nucleic acid molecule encoding said immunogen.

The invention further provides a kit of parts comprising a vaccine composition and a composition comprising a CD94/NKG2A and/or a CD94/NKG2B binding antibody or a CD94/NKG2A and/or a CD94/NKG2B binding part thereof, wherein said vaccine comprises an immunogen for eliciting an immune response against an antigen or a nucleic acid molecule encoding said immunogen.

Also provided is a use of a CD94/NKG2A and/or a CD94/NKG2B antibody or a CD94/NKG2A and/or a CD94/NKG2B binding part thereof and an immunogen for the production of an immune cell containing cell product for transplantation.

Also provided is a method for preparing an immune cell containing cell product comprising culturing a collection of cells comprising T-cells and/or NK-cells in the presence of an immunogen and a CD94/NKG2A and/or a CD94/NKG2B antibody or a CD94/NKG2A and/or a CD94/NKG2B binding part thereof, the method further comprising collecting T-cells and/or NK-cells after said culturing.

The invention further provides a method for stimulating an immune response in a subject comprising administering a vaccine and a CD94/NKG2A and/or a CD94/NKG2B binding antibody or a CD94/NKG2A and/or a CD94/NKG2B binding part thereof to the subject in need thereof, wherein said vaccine comprises an immunogen for eliciting an immune response against an antigen or a nucleic acid molecule encoding said immunogen.

The invention further provides a combination of a vaccine and a CD94/NKG2A and/or a CD94/NKG2B binding antibody or a CD94/NKG2A and/or a CD94/NKG2B binding part thereof for use in the treatment of a subject in need thereof, wherein said vaccine comprises anti-tumor lymphocytes; an immunogen for eliciting an immune response against an antigen; a nucleic acid molecule encoding said immunogen or a combination thereof.

The invention further provides a method for the treatment of an individual with cancer, the method comprising administering to the individual in need thereof a vaccine and a CD94/NKG2A and/or a CD94/NKG2B binding antibody or a CD94/NKG2A and/or a CD94/NKG2B binding part thereof, wherein the vaccine comprises anti-tumor lymphocytes; an immunogen for eliciting an immune response against an antigen; a nucleic acid molecule encoding said immunogen or a combination thereof.

### DETAILED DESCRIPTION OF THE INVENTION

A vaccine is a preparation comprising a biological molecule such as a protein, or a nucleic acid molecule encoding the protein, a carbohydrate, a lipid or a combination thereof that improves an immune response towards the biological molecule and/or cells containing the biological molecule. A vaccine typically, but not necessarily improves immunity towards a particular disease. A vaccine typically contains an immunogen or a nucleic acid molecule that codes for the immunogen, that resembles a disease-causing pathogen, protein, cell or part thereof. The immunogen stimulates the body's immune system to recognize the disease causing agent as foreign, destroy it, and keep a record of it, so that the immune system can more easily recognize and destroy or inactivate any of the of same disease causing agents that it later encounters.

There are prophylactic and therapeutic vaccines. The term vaccine typically refers to the product that is administered to the subject, i.e. including adjuvant (if any), carrier protein (if any), stabilizer, or other excipients. In the present invention the term vaccine includes the mentioned product but also includes preparations that contain the immunogen and/or nucleic acid molecule(s) that code for the immunogen, per se. The term vaccine as used herein is not limited to commercially available vaccines. The term vaccine as used herein does not imply that the preparation is effective in preventing disease or curing disease. The term vaccine includes all preparations that contain the immunogen and/or nucleic acid molecule(s) that code for the immunogen.

An antigen is any substance that may be specifically bound by components of the immune system (antibody, lymphocytes). Despite the fact that all antigens are recognized by specific lymphocytes or by antibodies, not every antigen can evoke an immune response. Those antigens that are capable of inducing an immune response are said to be immunogenic and are called immunogens in the present invention.

An immunogen is any antigen that is capable of inducing humoral and/or cell-mediated immune response rather than immunological tolerance. This ability is called immunogenicity. The immunogen is said to elicit an immune response against an antigen in a subject when the subject develops a humeral or cellular response to the immunogen upon is administration.

The term “immunogen” is defined herein as a complete antigen which is composed of the macromolecular carrier and one or more epitopes (determinants) that can induce immune response.

The macromolecular carrier and the one or more epitopes can be contained in a single molecule, such as a protein, be present in a particle such as a cell, or part or fragment thereof. The epitope may also be provided to a separate carrier. A non-limiting example is a hapten. Haptens are low-molecular-weight compounds that may be bound by antibodies, but cannot elicit an immune response. Consequently the haptens themselves are nonimmunogenic and they cannot evoke an immune response until they bind with a larger carrier immunogenic molecule. The hapten-carrier complex, unlike free hapten, can act as an immunogen and can induce an immune response.

The present invention provides means, methods and uses as described herein wherein the term vaccine is replaced by the phrase “immunogen or nucleic acid molecule encoding the immunogen”.

The NKG2 family of genes, designated NKG2A, C, D and E, was originally identified by screening a subtractive library enriched for NK- and T cell-specific transcripts. The NKG2A gene encodes two isoforms, NKG2A and NKG2B, with the latter lacking the stem region. Chromosomal mapping and analysis of the cDNA sequences showed that like CD94, the NKG2 genes are located in the NK complex on chromosome 12 and the proteins encoded by these genes are members of the C-type lectin family. NKG2A is a partner of CD94. NKG2A and CD94 form heterodimers which are expressed on the cell surface of NK cells and other immune cells. NKG2B also forms a heterodimer with CD94. The transmission of an inhibitory signal after CD94 cross-linking correlates with the expression of NKG2A by NK cell clones. The CD94/NKG2A heterodimer and the CD94/NKG2B heterodimer can deliver an inhibitory signal to NK and other CD94/NKG2A and/or CD94/NKG2B expressing immune cells, presumably mediated by the cytoplasmic domain of NKG2A/B (A. G. Brooks et al. (1997) J. Exp. Med. Volume 185, pp: 795-800). The term “CD94/NKG2A” refers to the heterodimer in humans and to the heterodimer of orthologs in other mammalian species. Specific mammalian orthologs may be known under different scientific names. The term as used herein encompasses such orthologs. The human CD94/NKG2A heterodimer and antibodies that bind to the human CD94/NKG2A heterodimer are preferred. In humans CD94 is also known as killer cell lectin-like receptor subfamily D, member 1 (KLRD1; UniGene 1777996). NKG2A/B is also known as killer cell lectin-like receptor subfamily C, member 1 (KLRC1; UniGene 903323). The term “CD94/NKG2B” refers to the heterodimer in humans and to the heterodimer of orthologs in other mammalian species. Specific mammalian orthologs may be known under different scientific names. The term as used herein encompasses such orthologs. The human CD94/NKG2B heterodimer and antibodies that bind to the human CD94/NKG2B heterodimer are preferred.

When reference is made to NKG2A/B the reference includes NKG2A, NKG2B or both.

A CD94/NKG2A/B binding antibody or a CD94/NKG2A and/or a CD94/NKG2B binding part thereof binds to the extra-cellular part of the CD94/NKG2A/B heterodimer receptor. An antibody typically binds a target via the antigen-binding site of the antibody. The antigen-binding site is typically formed by and present in the variable domain of the antibody. The variable domain contains the antigen-binding site. A variable domain that binds an antigen is a variable domain comprising an antigen-binding site that binds the antigen.

In one embodiment an antibody variable domain of the invention comprises a heavy chain variable region (VH) and a light chain variable region (VL). The antigen-binding site can be present in the combined VH/VL variable domain, or in only the VH region or only the VL region. When the antigen-binding site is present in only one of the two regions of the variable domain, the counterpart variable region can contribute to the folding and/or stability of the binding variable region, but does not significantly contribute to the binding of the antigen itself.

As used herein, antigen-binding refers to the typical binding capacity of an antibody to its antigen. An antibody that binds to CD94/NKG2A and/or CD94/NKG2B binds to CD94/NKG2A/B but under otherwise identical conditions, at least 100-fold lower to the CD94/NKG2C or CD94/NKG2D receptors of the same species. The epitope of the CD94/NKG2A antibody on CD94/NKG2A is typically present on the NKG2A part of the heterodimer. The epitope may also partly be on CD94. The epitope of the CD94/NKG2B antibody on CD94/NKG2B is typically present on the NKG2B binding part of the heterodimer. The epitope may also partly be on CD94. An antibody that binds NKG2A may also bind NKG2B, and vice versa. Considering that the CD94/NKG2A/B are cell surface receptors, the binding is typically assessed on cells that express the receptor(s). The antibodies of the present invention bind to the extra-cellular part of the CD94/NKG2A and/or the CD94/NKG2B heterodimer. Binding of an antibody to an antigen can be assessed in various ways. One way is to incubate the antibody with the antigen (preferably cells expressing the antigen), removing unbound antibody (preferably by a wash step) and detecting bound antibody by means of a labeled antibody that binds to the bound antibody.

Antigen binding by an antibody is typically mediated through the complementarity regions of the antibody and the specific three-dimensional structure of both the antigen and the variable domain allowing these two structures to bind together with precision (an interaction similar to a lock and key), as opposed to random, non-specific sticking of antibodies. As an antibody typically recognizes an epitope of an antigen, and as such epitope may be present in other compounds as well, antibodies according to the present invention that bind CD94/NKG2A may recognize other proteins as well, if such other compounds contain the same epitope. Hence, the term “binding” does not exclude binding of the antibodies to another protein or protein(s) that contain the same epitope. A CD94/NKG2A antibody as defined in the present invention typically does not bind to other proteins on the membrane of cells in a post-natal, preferably adult human. An antibody according to the present invention is typically capable of binding CD94/NKG2A with a binding affinity of at least 1×10e-6 M, as outlined in more detail below.

The term “interferes with binding” as used herein means that the antibody or NKG2A/B binding part thereof is directed to an epitope on CD94/NKG2A/B and the antibody or NKG2A/B binding part thereof competes with ligand for binding to CD94/NKG2A/B. HLA-E is a recognized ligand for the CD94/NKG2A/B heterodimer in humans. The mouse ortholog is generally known under the name Qa1. A CD94/NKG2A/B binding antibody or CD94/NKG2A and/or a CD94/NKG2B binding part thereof preferably interferes with binding of HLA-E to a CD94/NKG2A/B receptor. The antibody or binding part thereof may diminish ligand binding, displace ligand when this is already bound to CD94/NKG2A/B or it may, for instance through steric hindrance, at least partially prevent that ligand can bind to CD94/NKG2A/B.

The term “antibody” as used herein means a proteinaceous molecule, preferably belonging to the immunoglobulin class of proteins, containing one or more variable domains that bind an epitope on an antigen, where such domains are derived from or share sequence homology with the variable domain of an antibody. Antibodies for therapeutic use are preferably as close to natural antibodies of the subject to be treated as possible (for instance human antibodies for human subjects). Antibody binding can be expressed in terms of specificity and affinity. The specificity determines which antigen or epitope thereof is specifically bound by the binding domain. The affinity is a measure for the strength of binding to a particular antigen or epitope. Binding or specific binding, is defined as binding with affinities (KD) of at least 1×10e-6 M, more preferably 1×10e-7 M, more preferably higher than 1×10e-9 M. Typically, antibodies for therapeutic applications have affinities of up to 1×10e-10 M or higher. CD94/NKG2A/B binding antibodies may be monospecific antibodies or bi-specific antibodies. In a bi-specific antibody at least one of the VH/VL combinations binds CD94/NKG2A/B. Antibodies such the bispecific antibodies of the present invention typically comprise the constant domains of a natural antibody. An antibody of the invention is typically a full length antibody, preferably of the human IgG subclass. A CD94/NKG2A/B binding antibody of the present invention is preferably of the human IgG1 subclass. Such antibodies of the invention have good ADCC and/or CDCC properties. Such an antibody can be used to kill the CD94/NKG2A/B expressing cell thereby removing immune response dampening effects of these cells from the system. In a preferred embodiment the CD94/NKG2A/B binding antibody is of the human IgG4 subclass or another IgG subclass, such as IgG2 that does not exhibit ADCC or CDCC. Also derivatives of IgG1 are available that with reduced ADCC and/or CDCC. Such antibodies do not efficiently mark a bound cell for destruction. Such antibodies are typically preferred in the present invention as they at least reduce signaling of the CD94/NKG2A/B when bound.

In a preferred embodiment the CD94/NKG2A/B antibody reduces signaling of CD94/NKG2A/B on CD94/NKG2A/B-expressing natural killer cells. In a preferred embodiment the CD94/NKG2A/B antibody reduces ligand-induced signaling of CD94/NKG2A/B on CD94/NKG2A/B-expressing natural killer cells. In a human context the preferred ligand is HLA-E, preferably in the context of an HLA-E expressing cell. Ligand-induced receptor signaling is reduced by at least 20%, preferably at least 30, 40, 50 60, or at least 70% in a particularly preferred embodiment the ligand-induced receptor signaling is reduced by 80, more preferably by 90%. The reduction is preferably determined by determining a ligand-induced receptor signaling in the presence of a CD94/NKG2A/B binding antibody as referred to herein. The signaling is preferably compared with signaling in the absence of the antibody, under otherwise identical conditions. The conditions comprise at least the presence of an HLA-E ligand or, when applicable, ortholog thereof. The amount of ligand present is preferably an amount that induces half of the maximum signaling in a CD94/NKG2A/B positive cell line. Signaling is preferably determined by determining cell activation. Cell activation can be measured with proliferation, production of cytokines including IFN-gamma, or surface display markers including CD69 or CD137. In a preferred embodiment the CD94/NKG2A/B antibody or CD94/NKG2A and/or a CD94/NKG2B binding part thereof inhibits signaling of CD94/NKG2A/B on CD94/NKG2A/B-expressing natural killer cells. Inhibition of signaling implies a reduction of signaling by at least 90% preferably at least 95%. The reduction in signaling is preferably measured on NK-cells as a measure for activity of the antibody. An antibody that reduces signaling on NK-cells also reduces signaling on other CD94/NKG2A/B expressing immune cells.

In a preferred embodiment the CD94/NKG2A/B antibody or CD94/NKG2A and/or a CD94/NKG2B binding part thereof reduces signaling of CD94/NKG2A/B on CD94/NKG2A/B-expressing T-cells. In a preferred embodiment the CD94/NKG2A/B antibody or CD94/NKG2A and/or a CD94/NKG2B binding part thereof reduces ligand-induced signaling of CD94/NKG2A/B on CD94/NKG2A/B-expressing T-cells. In a human context the preferred ligand is HLA-E, preferably in the context of an HLA-E expressing cell. Ligand-induced receptor signaling is reduced by at least 20%, preferably at least 30, 40, 50 60, or at least 70% in a particularly preferred embodiment the ligand-induced receptor signaling is reduced by 80, more preferably by 90%. The reduction is preferably determined by determining a ligand-induced receptor signaling in the presence of a CD94/NKG2A/B binding antibody as referred to herein. The signaling is preferably compared with signaling in the absence of the antibody, under otherwise identical conditions. The conditions comprise at least the presence of an HLA-E ligand or, when applicable, ortholog thereof. The amount of ligand present is preferably an amount that induces half of the maximum signaling in a CD94/NKG2A/B positive cell line. Signaling is preferably determined by determining cell activation. Cell activation can be measured with proliferation, production of cytokines including IFN-gamma, or surface display markers including CD69 or CD137. In a preferred embodiment the CD94/NKG2A/B antibody inhibits signaling of CD94/NKG2A/B on CD94/NKG2A/B-expressing T-cells. Inhibition of signaling implies a reduction of signaling by at least 90% preferably at least 95%. The reduction in signaling is preferably measured on T-cells as a measure for activity of the antibody. An antibody that reduces signaling on T-cells also reduces signaling on other CD94/NKG2A/B expressing immune cells.

CD94/NKG2A/B antibodies or CD94/NKG2A and/or a CD94/NKG2B binding parts thereof that reduce and/or inhibit signaling can compete with the ligand for binding to the CD94/NKG2A heterodimer or not. In a preferred embodiment the CD94/NKG2A/B antibody or CD94/NKG2A and/or a CD94/NKG2B binding part thereof does not significantly compete with the ligand for binding to the CD94/NKG2A/B heterodimer. Competition of binding can be determined by binding studies of the antibody in the presence or absence of the ligand.

In a preferred embodiment the CD94/NKG2A antibody or CD94/NKG2A and/or a CD94/NKG2B binding part thereof competes for binding to CD94/NKG2A with antibody 2199 as described in EP2628753 (Novo Nordisk AS). In a preferred embodiment the antibody is the mentioned 2199 or humanized version thereof or a CD94/NKG2A and/or a CD94/NKG2B binding part thereof. In another preferred embodiment the CD94/NKG2A antibody or CD94/NKG2A and/or a CD94/NKG2B binding part thereof does compete with the ligand for binding to the CD94/NKG2A heterodimer. In a preferred embodiment the antibody or binding part thereof competes for binding to CD94/NKG2A with antibody 2270 as described in EP2628753 (Novo Nordisk AS). In a preferred embodiment the antibody is the mentioned 2270 or humanized version thereof.

An antibody that binds CD94/NKG2A or CD94/NKG2A binding part of such an antibody is preferred in the means, methods and uses of the present invention. An antibody or CD94/NKG2A binding part thereof, that binds to CD94/NKG2A preferably binds to CD94/NKG2A but under otherwise identical conditions, at least 100-fold lower to CD94/NKG2B.

The binding molecule can be an antibody. In the present invention an antibody is a full length antibody or a part thereof. Suitable parts retain the antigen binding capacity of the antibody in kind, not necessarily in amount. Suitable antibody parts are single chain Fv-fragments, monobodies, VHH, and Fab-fragments. A common denominator of such specific binding molecules is the presence of a heavy chain variable domain and for many also the corresponding light chain variable domain. A part of an antibody may contain further amino acid sequences such as, but not limited to, sequences to reduce the otherwise rapid clearance of such parts form the blood stream. A suitable carrier for single chain Fv fragment is among others human serum albumin. An antibody of the invention is preferably a “full length” antibody. The term ‘full length’ according to the invention is defined as comprising an essentially complete antibody, which however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH, and CL, VL. An antibody binds to antigen via the variable domains contained in the Fab portion, and after binding can interact with molecules and cells of the immune system through the constant domains, mostly through the Fc portion. The terms ‘variable domain’, ‘VH/VL pair’, ‘VH/VL’ are used herein interchangeably. Full length antibodies according to the invention encompass antibodies wherein mutations may be present that provide desired characteristics. Such mutations should not be deletions of substantial portions of any of the regions. However, antibodies wherein one or several amino acid residues are deleted, without essentially altering the binding characteristics of the resulting antibody are embraced within the term “full length antibody”. For instance, an IgG antibody can have 1-20 amino acid residue insertions, deletions or a combination thereof in the constant region. For instance, ADCC activity of an antibody can be improved when the antibody itself has a low ADCC activity, by slightly modifying the constant region of the antibody (Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489). On the other hand, ADCC activity can be reduced by modifying the constant region of the antibody.

Full length IgG antibodies are preferred because of their favorable half life and the need to stay as close to fully autologous (human) molecules for reasons of immunogenicity. In order to prevent any immunogenicity in humans it is preferred that the IgG antibody according to the invention is a human IgG4. In a preferred embodiment the IgG4 is engineered with such that it has reduced disulfide bond heterogeneity and/or increased Fab domain thermal stability (S. J Peters et al (2012). The J. of Biol. Chem. Vol. 287: pp. 24525-24533).

Antibodies may be derived from various animal species. Some antibodies have a murine background, at least with regard to the heavy chain variable region. It is common practice to humanize such e.g. murine heavy chain variable regions. There are various ways in which this can be achieved. It is possible to graft CDR into a human heavy chain variable region with a 3D-structure that matches the 3-D structure of the murine heavy chain variable region; one can deimmunize the murine heavy chain variable region, preferably by removing known or suspected T- or B-cell epitopes from the murine heavy chain variable region. The removal is typically by substituting one or more of the amino acids in the epitope for another (typically conservative) amino acid, such that the sequence of the epitope is modified such that it is no longer a T- or B-cell epitope.

Such deimmunized murine heavy chain variable regions are less immunogenic in humans than the original murine heavy chain variable region. Preferably a variable region or domain of the invention is further humanized, such as for instance veneered. By using veneering techniques, exterior residues which are readily encountered by the immune system are selectively replaced with human residues to provide a hybrid molecule that comprises either a weakly immunogenic or substantially non-immunogenic veneered surface. An animal as used in the invention is preferably a mammal, more preferably a primate, most preferably a human.

The concentration of immunogen in a vaccine is preferably between 1 ng/ml and 10 mg/ml, preferably between 10 ng/ml and 1 mg/ml, more preferably between 100 ng/ml and 100 mcg/ml, such as between 1 mcg/ml and 100 mcg/ml. The concentration is preferably at least 1 ng/ml to ensure that protein is in a concentration sufficient to exert its therapeutic effect when administered to an individual. The concentration should, however, preferably not exceed 10 mg/ml in order to prevent or reduce the occurrence of possible side effects associated with administration of said protein to a subject.

Nucleic acid encoding an immunogen in a vaccine may be RNA, DNA or analogue thereof. The nucleic acid molecule may be associated with virus proteins, typically a virus capsid for instance, for efficient delivery of the nucleic acid molecule to cells.

The combination of a vaccine and a CD95/NKG2A/B binding antibody or a CD94/NKG2A and/or a CD94/NKG2B binding part thereof may be present in one formula that is administered together to the subject. In one embodiment the invention therefore provides a pharmaceutical composition comprising a vaccine and a CD94/NKG2A/B binding antibody or a CD94/NKG2A and/or a CD94/NKG2B binding part thereof, wherein said vaccine comprises an immunogen for eliciting an immune response against an antigen or a nucleic acid molecule encoding said immunogen. The pharmaceutical composition preferably comprises an adjuvant and/or one or more suitable excipients such as a stabilizer, a buffer, a salt and the like. In a preferred embodiment the immunogen in the pharmaceutical composition is a tumor-antigen.

In a preferred embodiment the vaccine and antibody are in separate containers and administered separately to the subject. The vaccine and antibody may administered at essentially the same time, or sequentially. It is preferred that the antibody is administered prior to the vaccine or at essentially the same time. To this end the invention further provides a kit of parts comprising a vaccine composition and a composition comprising a CD94/NKG2A/B binding antibody or a CD94/NKG2A and/or a CD94/NKG2B binding part thereof, wherein said vaccine comprises an immunogen for eliciting an immune response against an antigen or a nucleic acid molecule encoding said immunogen. In case of the vaccine composition the composition may further comprise an adjuvant. Both composition may further comprise one or more suitable excipients such as a stabilizer, a buffer, a salt and the like.

The subject to be treated is preferably a human subject.

It is preferred that the treatment comprises a cancer treatment. In this embodiment it is preferred that the vaccine is a cancer vaccine. In this embodiment it is preferred that the immunogen is a tumor-antigen, preferably a tumor-specific antigen.

A tumor antigen is an antigenic substance produced in tumor cells. The host comprising the tumor may elicit an immune response to the antigen, or the antigen may be immunogenic upon vaccination of the host, preferably by means of a method of the invention. Tumor antigens are useful tumor markers in identifying tumor cells with diagnostic tests and are used in cancer therapy. Since the discovery of the first tumor antigens many different further antigens have been identified. Several mechanisms have been identified that can result in the production of a tumor-antigen by a tumor cell. Normal proteins in the body are typically, though not necessarily, not antigenic because of self-tolerance, a process in which self-reacting cytotoxic T lymphocytes (CTLs) and autoantibody-producing B lymphocytes are culled “centrally” in primary lymphatic tissue (BM) and “peripherally” in secondary lymphatic tissue (mostly thymus for T-cells and spleen/lymph nodes for B cells). Thus any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation, different processing, different folding or the like.

Tumor antigens can be broadly classified into two categories based on their pattern of expression: Tumor-Specific Antigens (TSA), which are present only on tumor cells and not on any other cell in the subject at the time that he has the tumor and Tumor-Associated Antigens (TAA), which are present on tumor cells and also some normal cells. Tumor-specific antigens may (have been) expressed in the subject at times different than when having the tumor. For instance, some tumor-specific antigens are expressed during embryogenesis. Various classes of tumor antigens are presently recognized. Products of Mutated Oncogenes and Tumor Suppressor Genes; Products of Other Mutated Genes Overexpressed or Aberrantly Expressed Cellular Proteins; Tumor Antigens Produced by Oncogenic Viruses; Oncofetal Antigens; Altered Cell Surface Glycolipids and Glycoproteins; Cell Type-Specific Differentiation Antigens. This list is not intended to be limitative.

Any protein produced in a tumor cell that has an abnormal structure due to mutation; different post-translational modification; folding and the like can act as a tumor antigen. Such abnormal proteins can be produced as a result of mutation of the concerned gene or different amount of production or different processing. Mutation of protooncogenes and tumor suppressors which lead to abnormal protein production can be the cause of the tumor and such abnormal proteins are called tumor-specific antigens. Examples of tumor-specific antigens include the abnormal products of ras and p53 genes. Other examples include tissue differentiation antigens, mutant protein antigens, oncogenic viral antigens, cancer-testis antigens and vascular or stromal specific antigens. Tissue differentiation antigens are those that are specific to a certain type of tissue. Mutant protein antigens are likely to be more specific to cancer cells because normal cells shouldn't contain these proteins. Normal cells will display the normal protein antigen on their MHC molecules, whereas cancer cells will display the mutant version. Some viral proteins are implicated in forming cancer (oncogenesis), and some viral antigens are also cancer antigens. Cancer-testis antigens are antigens expressed primarily in the germ cells of the testes, but also in fetal ovaries and the trophoblast. Some cancer cells aberrantly express these proteins and therefore present these antigens, allowing attack by T-cells specific to these antigens. Example antigens of this type are CTAG1B and MAGEA1.

Proteins that are normally produced in very low quantities but whose production is dramatically increased in tumor cells, trigger an immune response. An example of such a protein is the enzyme tyrosinase, which is required for melanin production. Normally tyrosinase is produced in minute quantities but its levels are very much elevated in melanoma cells.

Oncofetal antigens are another important class of tumor antigens. Examples are alphafetoprotein (AFP) and carcinoembryonic antigen (CEA). These proteins are normally produced in the early stages of embryonic development and disappear by the time the immune system is fully developed. Thus self-tolerance does not develop against these antigens.

Abnormal proteins are also produced by cells infected and transformed by oncoviruses, e.g. EBV, HBV, HCV, and HPV. Cells infected by these viruses contain viral RNA and/or DNA which is transcribed and the resulting protein produces an immune response.

In addition to proteins, other substances like cell surface glycolipids and glycoproteins may also have an abnormal structure in tumor cells and could thus be targets of the immune system.

Tumor-antigens and their use in vaccines for the treatment of cancer are reviewed among others in Melief et al (J. of Clinical Investigation 2015; Vol 9: pp 3401-3412) and in Lampen and van Hall (Current opinion in Immunology 2011; Vol 23: pp 293-298). The described means and methods for preparing and using tumor-antigens are included by reference herein.

In one embodiment the vaccine comprises cells comprising the immunogen. In a preferred embodiment the cells comprise a tumor-antigen, preferably a tumor-specific antigen. In one embodiment the vaccine comprises tumor cells. The cells in a vaccine can be live cells, however, more commonly the cells are inactivated prior to incorporation into the vaccine, or prior to administration to the subject. Various method of inactivation of cells exist such as but not limited to formaldehyde or irradiation.

In the context of tumor vaccination it was found that the number of CD94/NKG2A expressing cells increases in the tumor upon providing the vaccine. The number of C94/NKG2A expressing NK-cells increases. In particular the number of CD94/NKG2A expressing T-cells increases. It was found that a substantial fraction of the CD94/NKG2A expressing T-cells do not express CTLA4, PD-1, or TIM3. It was found that the expression levels of the NKG2A ligand Qa-1 is increased in the tumor upon vaccination. In a preferred embodiment a combination of a vaccine and a CD94/NKG2A binding antibody further comprises at least one antibody selected from a CTLA4-binding antibody, a PD-1 binding antibody, a PD-L1 binding antibody; a LAG-3 binding antibody; a VISTA antibody and a TIM3 binding antibody or a antigen binding part of said antibody. The antibody or antigen binding part thereof preferably inhibits signaling of the CTLA4, PD-1, PD-L1, LAG, VISTA and/or TIM3. Various CTLA4, PD-1, PD-L1, LAG, VISTA and/or TIM3 signaling inhibiting antibodies are known in the art. In a preferred embodiment a combination of a vaccine and a CD94/NKG2A binding antibody or a CD94/NKG2A and/or a CD94/NKG2B binding part thereof further comprises at least one antibody selected from a CTLA4-binding antibody, a PD-1 binding antibody and a TIM3 binding antibody or an antigen binding part thereof. Combination with one or more of such antibodies or antigen binding parts thereof with a CD94/NKG2A/B binding antibody or a CD94/NKG2A and/or a CD94/NKG2B binding part thereof as described herein exhibits an improved effect. Without being bound by theory it is believed that this is due to the significant number of CD94/NKG2A/B expressing T-cells that do not significantly express CTLA4, PD-1 or TIM3.

The subject can be a subject infected with a pathogen. The subject can also be, among others a subject that has cancer. In a preferred embodiment the subject is a cancer patient. The cancer of the subject is preferably a solid cancer. The cancer is preferably ovarian carcinoma, head&neck carcinoma, melanoma, cervical carcinoma, pancreatic carcinoma, renal cell carcinoma, lung carcinoma, prostate carcinoma, virus induced carcinoma or colorectal carcinoma. This includes both the primary tumor and/or metastasis or pre-stage hyperplasia of the mentioned cancers. Virus induced carcinoma comprises among others carcinoma induced by human papilloma virus, hepatis B virus, hepatis C virus and Epstein barr virus (resp. HPV, HBV, HCV, EBV).

The invention further provides a use of a CD94/NKG2A/B antibody or a CD94/NKG2A and/or a CD94/NKG2B binding part thereof and an immunogen for the production of an immune cell containing cell product for transplantation. Also provided is a method for preparing an immune cell containing cell product comprising culturing a collection of cells comprising T-cells and/or NK-cells in the presence of an immunogen and a CD94/NKG2A/B antibody or a CD94/NKG2A and/or a CD94/NKG2B binding part thereof, the method further comprising collecting T-cells and/or NK-cells after said culturing. Immune cells can be produced in vitro in a culture of T-cells and/or NK-cells together with antigen-presenting cells and an immunogen. The immunogen can be provided as such. Antigen of the immunogen will be presented by the antigen-presenting cell. In a preferred embodiment the culture comprises cancer cells, or parts thereof comprising the immunogen. Suitable immune cells production methods are among others described in the following documents and references therein: Exploiting the curative potential of adoptive T-cell therapy for cancer. Hinrichs C S, Rosenberg S A. Immunol Rev. 2014 January; 257(1):56-71. doi: 10.1111/imr.12132. Adoptive cell transfer: a clinical path to effective cancer immunotherapy. Rosenberg S A, Restifo N P, Yang J C, Morgan R A, Dudley M E. Nat Rev Cancer. 2008 April; 8(4):299-308. doi: 10.1038/nrc2355. Clinical production and therapeutic applications of alloreactive natural killer cells. McKenna D H, Kadidlo D M, Cooley S, Miller J S. Methods Mol Biol. 2012; 882:491-507. doi: 10.1007/978-1-61779-842-9_28.

The invention also provides a method for stimulating an immune response in a subject comprising administering a vaccine and a CD94/NKG2A/B binding antibody or a CD94/NKG2A and/or a CD94/NKG2B binding part thereof to the subject in need thereof, wherein said vaccine comprises an immunogen for eliciting an immune response against an antigen or a nucleic acid molecule encoding said immunogen. The vaccine and the CD94/NKG2A/B binding antibody or a CD94/NKG2A and/or a CD94/NKG2B binding part thereof are provided/administered essentially at the same time.

The invention further provides a combination of an immune cell transplant and a CD94/NKG2A and/or a CD94/NKG2B binding antibody or a CD94/NKG2A and/or a CD94/NKG2B binding part thereof, for use in the treatment of a subject in need thereof. The combination preferably further comprises a vaccine that comprises an immunogen for eliciting an immune response against an antigen or a nucleic acid molecule encoding said immunogen. The immune cell transplant is preferably an immune cell containing cell product as described herein above. Immune cell transplants are presently mostly used in the treatment of subjects with cancer. Immune cell transplants can comprise a collection of cells comprising T-cells and/or NK-cells. Means and methods for preparing T-cell transplants and treatment of a subject therewith are among others described in Rosenberg and Restifo (2015; Science Vol 348:pp 62-68). This reference and the references cited therein are incorporated by reference herein. Cells in the immune cell transplant are preferably tumor-reactive lymphocytes, preferably CD8⁺ T-cells. Such cells can be naturally tumor-reactive or be provided with (additional) tumor-reactivity through genetic modification. The modification typically involves heterologous expression of a tumor-specific T-cell receptor or so-called chimeric antigen receptors (CARs) as for instance described in the Rosenberg Restifo reference cited herein above. Immune cell transplants are also referred to as adoptive cell therapy. Adoptive cell therapy in the present invention is preferably used in the treatment of cancer. Preferably in the treatment of melanoma, virus-induced cancers, ovarian cancer, lung cancer, colorectal cancer, pancreatic cancer, lymphoma, leukemia, bile duct cancer and neuroblastoma.

### BRIEF DESCRIPTION OF THE DRAWINGS

FIG. 1. In VIN lesions, NKG2A associates with better clinical outcome.

A. Immunofluorescence tissue section stainings of CD3 (red) and NKG2A (green). NKG2A expression on CD8 T cells and NK cells is visualized in YIN lesions.

B. Number of NKG2A⁺ T cells were determined by tissue section stainings and divided by number of total T cells (‘CD3⁺NKG2A−’). This ratio had prognostic value in this cohort of YIN patients for recurrence free survival time. The expression of inhibitory receptors on T cells in malignancy are of prognostic value and seem to indicate an activated state of local T cells.

FIG. 2. Expression of NKG2A on CD8 T cells from tumor infiltrating lymphocytes of Head&Neck squamous cell carcinoma (HNSCC).

A. Frequency of CD8 T cells expressing CD94 and NKG2A in the blood of healthy subjects is around 5%. This frequency is much higher in TIL of HNSCC samples.

B. Flow cytometry plots of 8-color staining panel designed to determine profiles of inhibitory receptor co-expression on lymphocyte subsets. CD94⁺NKG2A⁺ CD8⁺ T cells are further gates to analyse the expression of other inhibitory receptors TIM-3 and PD-1. Inhibitory receptors are mosaically expressed on lymphocytes, creating several different subsets with increasing number of receptors.

C. Representation of data from figure B., indicating frequencies of CD8 T cells that express none, single or multiple inhibitory receptors in TIL of HNSCC patient samples (right pie-charts) or PBMC of healthy subjects (left pie-charts). Approximately 30% of NKG2A⁺ CD8 T cells in these cancers do not co-express TIM-3, PD-lor CTLA-4.

FIG. 3. NKG2A and Qa-1 (=mouse HLA-E) are strongly increased after immunotherapy.

A. Treatment scheme of B16F10 melanomas. Tumor-specific pmel T cells with transgenic TCR for gp100 were infused and in vivo activated by two vaccinations with synthetic long peptides

B. Tumor growth curves and survival curves are shown for non-treated and immunotherapy-treated groups of tumor-bearing mice.

C. Expression levels of Qa-1 (=mouse HLA-E) on B16F10 melanoma cells that were removed from the mice, dispersed and stained for flow cytometry. Immunotherapy led to strongly increased levels of Qa-1.

D. Flow cytometry of intratumoral CD8 T cells (CTL) and NK cells for expression of the inhibitory receptor CD94/NKG2A. Spleen-derived lymphocytes were taken along as control staining. On average 60% of CTL expressed the inhibitory receptor when mice had been treated with immunotherapy. Tumors were removed after outgrowth to maximal sizes.

FIG. 4. NKG2A and Qa-1 (=mouse HLA-E) are strongly increased after immunotherapy.

A. Treatment scheme of HPV-induced TC-1 carcinomas. Tumor-bearing mice were vaccinated once with an HPV comprising synthetic long peptide in mineral oil.

B. Tumor growth curves and survival curves are shown for non-treated and immunotherapy-treated groups of tumor-bearing mice.

C. Expression levels of Qa-1 (=mouse HLA-E) on TC-1 carcinoma cells that were removed from the mice, dispersed and stained for flow cytometry. Immunotherapy led to strongly increased levels of Qa-1.

D. Quantification of the data shown in panel C. Mean fluorescence values are depicted with standard error of the mean.

E. Flow cytometry of intratumoral CD8 T cells (CTL) and NK cells for expression of the inhibitory receptor CD94/NKG2A. Spleen-derived lymphocytes were taken along as control staining. On average 75% of CTL expressed the inhibitory receptor when mice had been treated with immunotherapy. Tumors were removed at day 19 of tumor inoculation.

F. Quantification of data shown in panel E. Frequencies of NKG2A⁺ cells of all CTL and of all NK cells.

G. Expression of NKG2A on CTL is associated with tumor-specificity as measured with HPV16 E7-tetramers (‘HPV™’).

H. Therapeutic vaccination with synthetic long peptides recruits CTL and NK cells to the site of the tumor.

FIG. 5. Blocking of the inhibitory receptor NKG2A on CD8 T cell clones increases reactivity in vitro.

A. Experimental set up. Antigen-specific CD8 T cell clones were incubated with anti-NKG2A antibodies (20d5 for mouse; 2199 for human) and incubated with peptide-loaded antigen-presenting cells that express high levels of the CD94/NKG2A ligand (LPS-blasts for mouse; B-LCL cells for human). Reactivity was measured after 20 h incubations time (IFNy release for mouse; CD137 display for human).

B. Mouse CD8 T cell clone expresses uniformly CD94 and NKG2A chains and were incubated with control peptide or cognate stimulating peptide in the presence of increasing concentration of blocking NKG2A antibody. T cell reactivity was measured by IFNy release as determined in ELISA. Strongly increased CTL reactivity can be observed by blocking NKG2A.

C. Human CD8 T cell clone displayed heterogeneous expression of CD94 and NKG2A. This mixed population was incubated with peptide loaded B-LCL cells and reactivity of the CTL at a per cell basis was measured in flow cytometry by induction of CD137 (4-1BB) at the cell surface. The reactivity of NKG2A-expressing CTL can be enhanced by the blocking antibody, but not NKG2A-negative CTL.

FIG. 6: Staining of tumor infiltrating CD8⁺ T cells, β2-microglobulin, HLA-A, HLA-B/C and HLA-E in pulmonary adenocarcinoma.

Examples of high (A) and low (B) stromal and intraepithelial CD8⁺ T cell infiltration;

tumor with high β2-microglobulin expression (C); examples of HLA-A (D), HLA-B/C (E) and HLA-E (F) staining Original magnification ×200.

FIG. 7. Association of CD8⁺ T cell infiltration and HLA expression with OS.

Survival curves of patients with low or high intraepithelial CD8+ T cells (A); stromal CD8+ T cells (B) and total CD8+ T cells (C)

Survival curves are presented for functional (i.e. positive staining for both HLA and β2-M) expression of HLA-A (D), HLA-B/C (E) and HLA-E (F). A significant correlation (p=0.042) was observed between low HLA-E expression and improved survival (F).

FIG. 8. Effect of classical HLA class I expression and CD8⁺ T cell infiltration on OS.

(A,B) Total CD8⁺ T cell infiltration in the context of HLA-A expression did not have prognostic impact.

(C,D) HLA-B/C positive tumors with high total CD8⁺ T cell infiltration showed better OS (D) whereas this effect was not observed in tumors with low HLA-B/C expression (C).

(E,F) Improved OS was established for tumors with high expression for both HLA-A and HLA-B/C when high total CD8⁺ T cell infiltration was present (F) while conversely this effect was not seen in low HLA-A and HLA-B/C expressing tumors (E).

FIG. 9. Prognostic benefit in HLA-E negative tumors with high CD8⁺ T cell infiltration.

(A,B) In tumors with low HLA-E expression, a high stromal CD8+ T cell infiltration was strongly associated with a better OS (A). Interestingly, the clinical benefit of a high stromal CD8⁺ T cell infiltrate was neutralized by high HLA-E expression (B). (C,D) Conversely, in patients with high stromal CD8⁺ T cell influx, a high HLA-E expression led to worse OS (C). In patients with low presence of stromal CD8⁺ T cells, HLA-E expression had no effect on OS (D).

FIG. 10. Tertile based grouping of stromal CD8+ T cells and influence on OS.

Stromal CD8+ T cell infiltration as a single determinant had a positive impact on clinical outcome but nearly missed statistical significance (FIG. 7B, log-rank test p=0.068). However, when CD8⁺ T cell counts/mm2 tumor were dichotomized based on tertiles instead of the mean, a significant effect was observed for patients with high (i.e. categorized in the middle and upper tertile) presence of stromal CD8+ T cells in the primary tumor (log-rank test p=0.046).

FIG. 11. HLA expression and its relation with total CD8+ T cell infiltration in the primary tumor.

A significant relation (Mann-Whitney U test, p<0.05) exists between high numbers of CD8⁺ T cells and classical HLA-A as well as HLA-B/C, but not for non-classical HLA-E.

### EXAMPLES

### Example 1

### Materials and Methods

### Flow Cytometry of Tumor Infiltrating Lymphocytes

Primary resected human tumors were minced and digested with gentleMACS. Tumor-infiltrating lymphocytes were expanded with IL-2 for 7 days, before immune-phenotyping by flow cytometry. The following anti-human antibodies were used, anti-CD3 (DAKO; clone UCHT1), anti-CD4 (BD; clone RPA-T4), anti-CD8 (BD; SK1), anti-CD56 (BD; clone B159), anti-CD94 (R&D systems; clone 131412), anti-NKG2A (Beckman Coulter; clone z199), anti-CTLA-4 (BD; clone BN13), anti-PD1 (Biolegend; clone EH12.2H7), anti-TIM3 (Biolegend; clone F38-2E2), anti-CD69 (BD; clone L78), and anti-CD137 (BD; 4B4-1). Samples were acquired with Fortessa flow cytometer (BD Biosciences) and analyzed with FlowJo software (TreeStar). Multi-parameter flow-cytometry data from Flowjo software was imported into SPICE software for multivariate analysis (Roederer 2011 Cytometry A).

Mouse tumor cells and infiltrating lymphocytes were isolated from primary tumors when tumors exceeded 1000 mm³ (B16 melanoma) and day 19 after tumor challenge (TC-1). TC-1 tumors were flushed before digestion. Subsequently, resected tumors were minced and digested using Liberase (Roche). Splenocytes were obtained after red blood cell lysis. Surface antigens were stained after Fc Block (BD; clone 2.4g2) using fluorescently labeled antibodies anti-CD45.2 (Biolegend; clone 104), anti-CD3 (Biolegend; clone 145-2C11), anti-CD4 (eBioscience; clone GK1.5), anti-CD8 (eBioscience; clone 53-6.7), anti-NK1.1 (Biolegend; clone PK136), anti-CD94 (eBioscience; clone 18D3), anti-NKG2A/C/E (BD; clone 20D5), anti-NKG2A (Biolegend; clone 16A11), and anti-Qa1 (BD; clone 6A8.6F10.1A6). MHC-I-tetramers containing the immunodominant peptide from HPV16 E7 (aa49-57) in was produced in-house. Samples were acquired with Fortessa flow cytometer (BD Biosciences) and analyzed with FlowJo software (TreeStar).

### NKG2A Blocking Assay

For blocking NKG2A receptor on human immune cells, influenza M1-specific CD8 T-cells were isolated from a HLA-A2 positive donor using magnetic activated cell sorting, using PE-labeled HLA-A2 tetramers containing the M1-derived peptide GILGFVFTF. These influenza-specific CD8 line was expanded in vitro as described earlier (Influenza matrix 1-specific human CD4⁺ FOXP3⁺ and FOXP3(−) regulatory T cells can be detected long after viral clearance. Piersma S J, van der Hulst J M, Kwappenberg K M, Goedemans R, van der Minne C E, van der Burg S H. Eur J Immunol. 2010 November; 40(11):3064-74. doi: 10.1002/eji.200940177). For NKG2A-blocking experiments, 100,000 M1-specific CD8 T cells were cocultured with 10,000 HLA-A2+B-LCL and increasing concentrations of z199 antibody (Beckman Coulter). After 2 hours pre-incubation M1 peptide was added and co-incubated overnight. Subsequently, the cells were stained with fluorescently labelled antibodies, measured by flow cytometry and analysed for expression of CD137 as a marker of T cell activation.

For blocking NKG2A receptor on mouse immune cells, CTL clone specific for the Trh4 antigen were cultured as described before (Peptide transporter TAP mediates between competing antigen sources generating distinct surface MHC class I peptide repertoires. Oliveira C C, Querido B, Sluijter M, Derbinski J, van der Burg S H, van Hall T. Eur J Immunol. 2011 November; 41(11):3114-24. doi: 10.1002/eji.201141836). For anti-body blocking, 2,000 CTL per well were pre-treated with 20D5 hybridoma supernatant for 1 hour, hereafter 5,000 cell/well peptide-loaded LPS-blasts were added as target cells. Culture supernatant was collected after 24-hour incubation. IFN-γ ELISA was performed on culture supernatant as previously described (Peptide transporter TAP mediates between competing antigen sources generating distinct surface MHC class I peptide repertoires. Oliveira C C, Querido B, Sluijter M, Derbinski J, van der Burg S H, van Hall T. Eur J Immunol. 2011 November; 41(11):3114-24. doi: 10.1002/eji.201141836). Data shown represent mean values obtained from triplicate test-wells, and the error bars represent standard deviation of these values.

### Mice, Cell Lines and Reagents

C57BL/6jico mice were purchased from Charles River (Lille, France) and used at 8 weeks of age. Pmel-1 TCR transgenic mice (Thy1.1 background) harbor the gp100₂₅₋₃₃/D^{b}-specific receptor were bred and housed in the animal facility of the Leiden University Medical Center under specific pathogen-free conditions. Experiments were approved by the local university committee for the care of laboratory animals (Dier Experimenten Commissie), in accordance with guidelines of the National Institutes of Health. B16F10 melanoma cell line was originally obtained from the American Type Culture Collection and maintained in tissue culture as described in (Peptide vaccination after T-cell transfer causes massive clonal expansion, tumor eradication, and manageable cytokine storm Ly L V, Sluijter M, Versluis M, Luyten G P, van Stipdonk M J, van der Burg S H, Melief C J, Jager M J, van Hall T. Cancer Res. 2010 Nov. 1; 70(21):8339-46. doi: 10.1158/0008-5472.CAN-10-2288). TC-1 cancer cell line contains the HPV16 E6 and E7 oncogenes and was obtained from TC Wu (Johns Hopkins Medical Institute, Baltimore, USA).

### Tumor Models

B16F10 melanoma model. A lethal dose of 3×10⁴ B16F10 melanoma cells was injected s.c. in syngeneic C57BL/6 mice. Previously established protocol for transfer of pmel-1 T cells and vaccination with 20-mer long gp100 peptide was applied (Peptide vaccination after T-cell transfer causes massive clonal expansion, tumor eradication, and manageable cytokine storm. Ly L V, Sluijter M, Versluis M, Luyten G P, van Stipdonk M J, van der Burg S H, Melief C J, Jager M J, van Hall T. Cancer Res. 2010 Nov. 1; 70(21):8339-46. doi: 10.1158/0008-5472.CAN-10-2288). HPV16 positive TC-1 model. Tumor cells were injected s.c. (1×10⁵) in syngeneic C57BL/6 mice. Vaccination with long synthetic peptide emulsified in IFA was performed at day 8 after tumor inoculation as previously described (Vaccine-induced effector-memory CD8⁺ T cell responses predict therapeutic efficacy against tumors. van Duikeren S, Fransen M F, Redeker A, Wieles B, Platenburg G, Krebber W J, Ossendorp F, Melief C J, Arens R. J Immunol. 2012 Oct. 1; 189(7):3397-403). Only one vaccination was applied. Tumor growth was monitored twice a week by measurement with a caliper in three dimensions.

### Results & Discussion

### The Inhibitory Receptor CD94/NKG2A as a Marker for Activated T Cells.

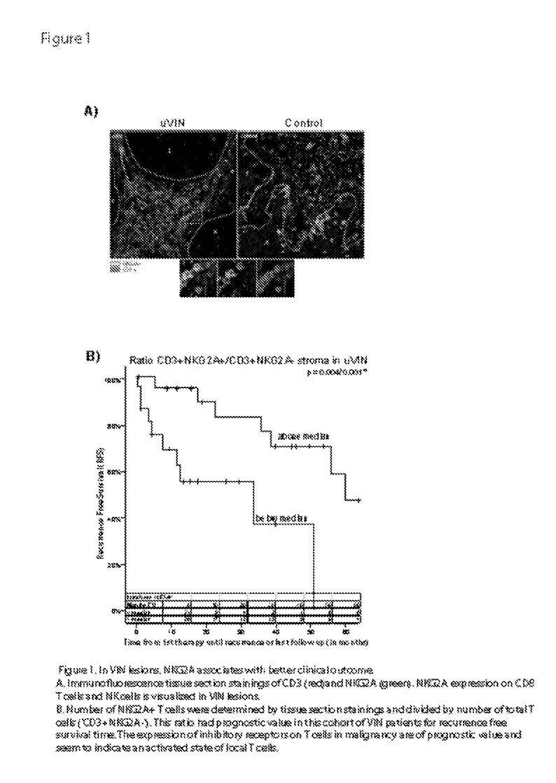

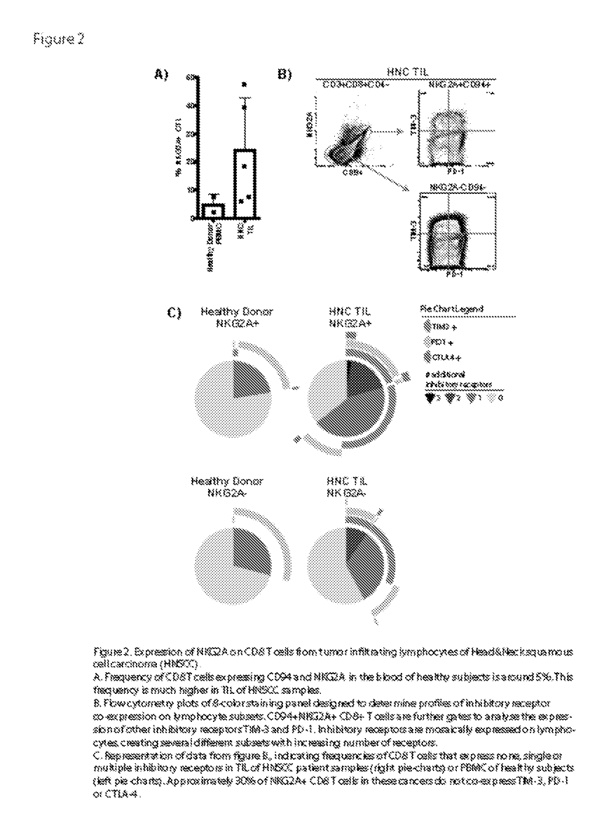

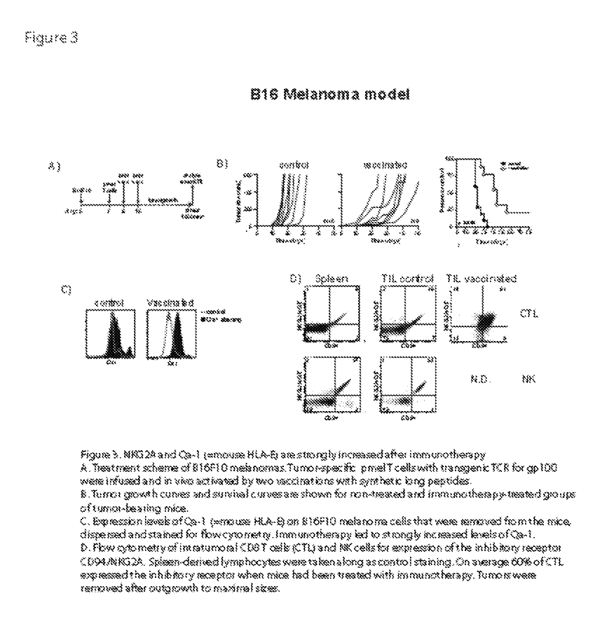

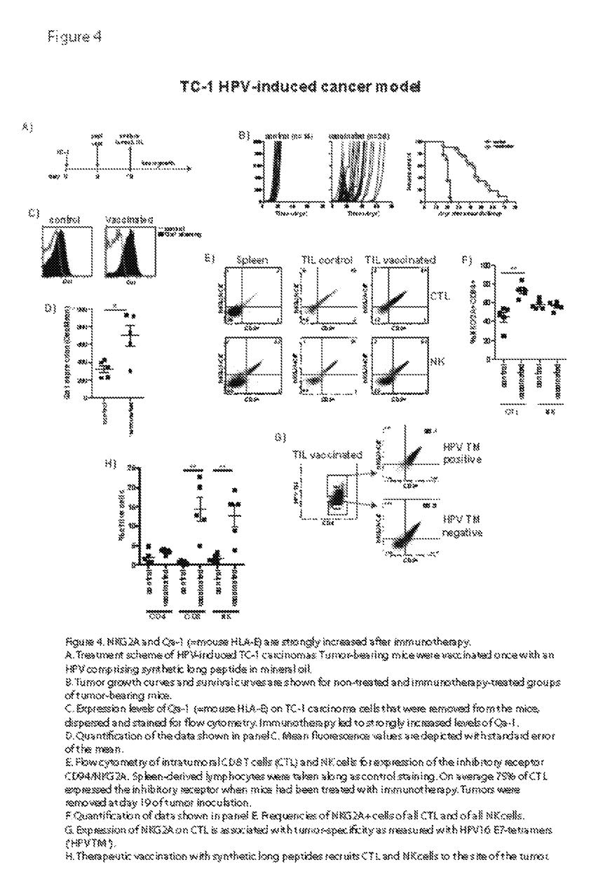

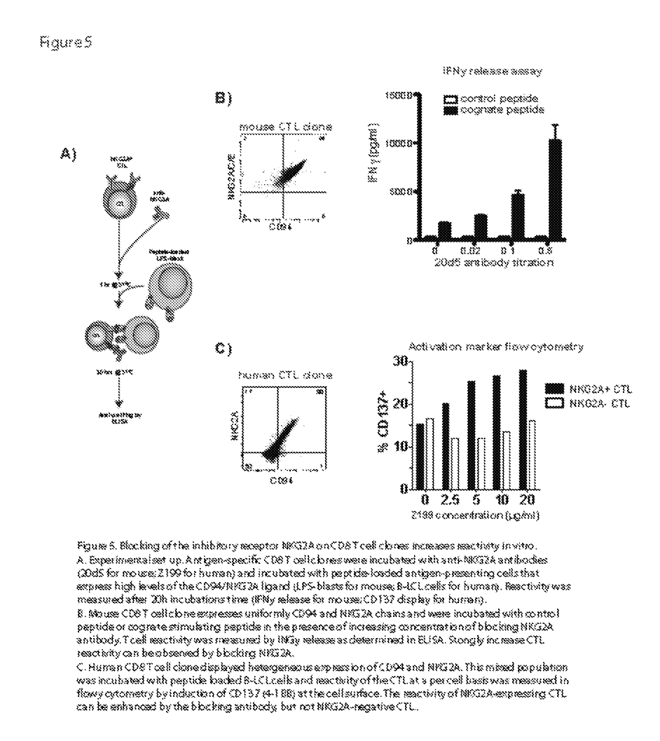

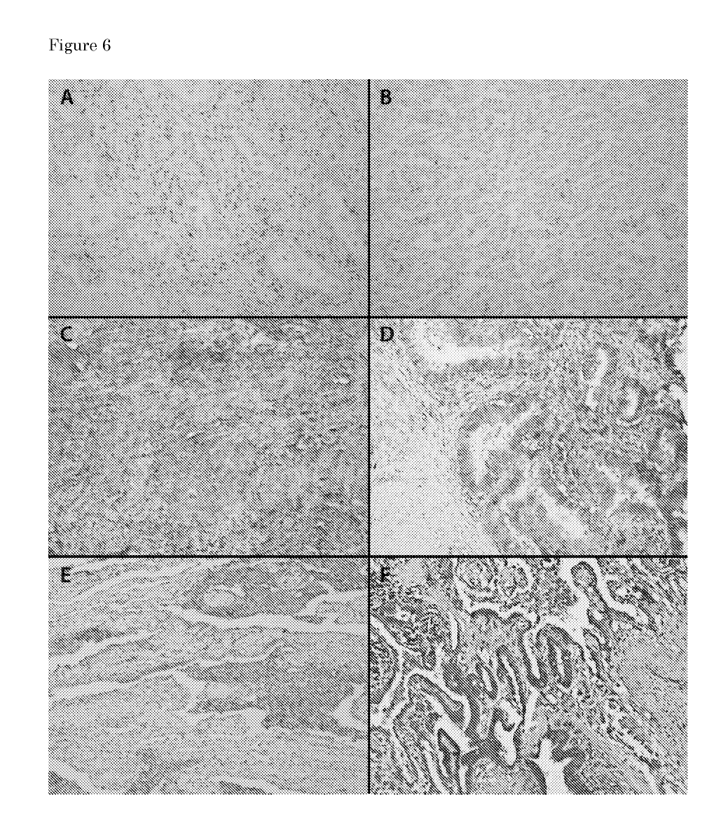

Initially, the expression of inhibitory receptors, including PD-1 and TIM-3, by T cells was thought to identify functionally ‘exhausted’ T cells. However, this concept has been refuted by studies showing that such inhibitory markers are predominantly expressed on activated CTL as part of normal immune regulation (Gros A, Robbins P F, Yao X, Li Y F, Turcotte S, Tran E, Wunderlich J R, Mixon A, Farid S, Dudley M E et al: PD-1 identifies the patient-specific CD8⁺ tumor-reactive repertoire infiltrating human tumors. In: J Clin Invest. 2014. Legat A, Speiser D E, Pircher H, Zehn D, Fuertes Marraco S A: Inhibitory Receptor Expression Depends More Dominantly on Differentiation and Activation than ‘Exhaustion’ of Human CD8 T Cells. In: Front Immunol. vol. 4; 2013: 455). Inhibitory receptors on activated T cells is thus not limited to situations of chronic stimulation, but merely reflect an antigen-experienced status. These receptors may even be used to enrich effective tumor-specific CTL for successful adoptive T cell therapy (Inozume T, Hanada K-I, Wang Q J, Ahmadzadeh M, Wunderlich J R, Rosenberg S A, Yang J C: Selection of CD8⁺PD-1⁺ lymphocytes in fresh human melanomas enriches for tumor-reactive T cells. In: J Immunother. vol. 33; 2010: 956-964). NKG2A has been shown to become expressed on CTL after TCR engagement (Jabri B, Selby J M, Negulescu H, Lee L, Roberts A I, Beavis A, Lopez-Botet M, Ebert E C, Winchester R J: TCR specificity dictates CD94/NKG2A expression by human CTL. In: Immunity. vol. 17; 2002: 487-499), underlining that this receptor is part of the normal regulatory feedback mechanisms of bona fide CTL. We have determined the infiltration of NKG2A⁺ T cells in 43 YIN lesions by immunofluorescence using an antibody to CD3⁺ (anti-CD3, rabbit, clone ab828; Abcam 1:100) and to NKG2A (anti-NKG2A, goat, clone N19; Santa Cruz 1:50) (FIG. 1A). Considerable intraepithelial and stromal infiltration of NKG2A⁺ T cells was observed in these malignancies. Importantly, enumeration of NKG2A⁺ T cells as a proportion of all infiltrating T cells revealed an association with clinical outcome. Extended recurrence free survival times were observed for those lesions with higher frequencies of NKG2A⁺ T cells, supporting the notion that this inhibitory receptor reflects activated T cells (FIG. 1B). Determination of TIM-3 expression yielded a very comparable profile (not shown). Therefore, NKG2A is absolutely a serious member of the inhibitory receptor family found on activated T cells and which can be targeted with blocking antibodies to release the full power of tumor-reactive T cells.

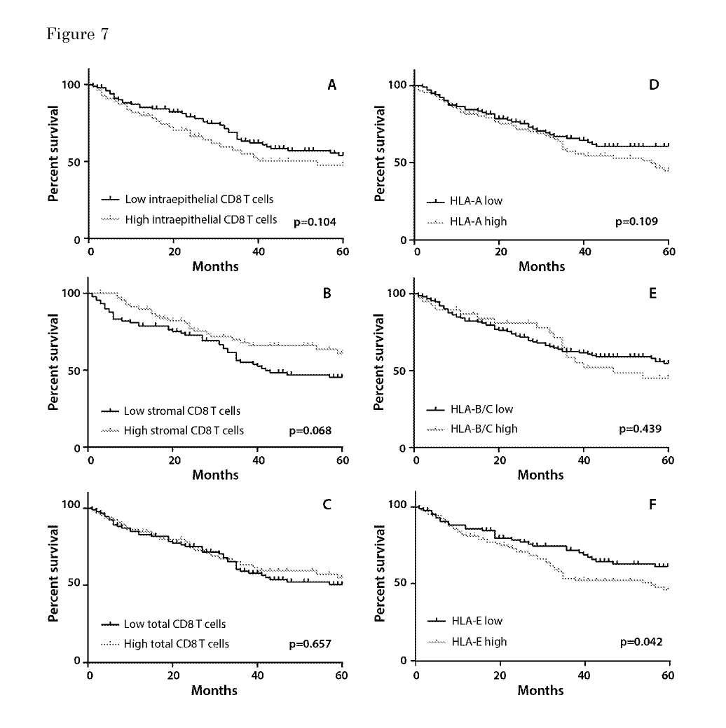

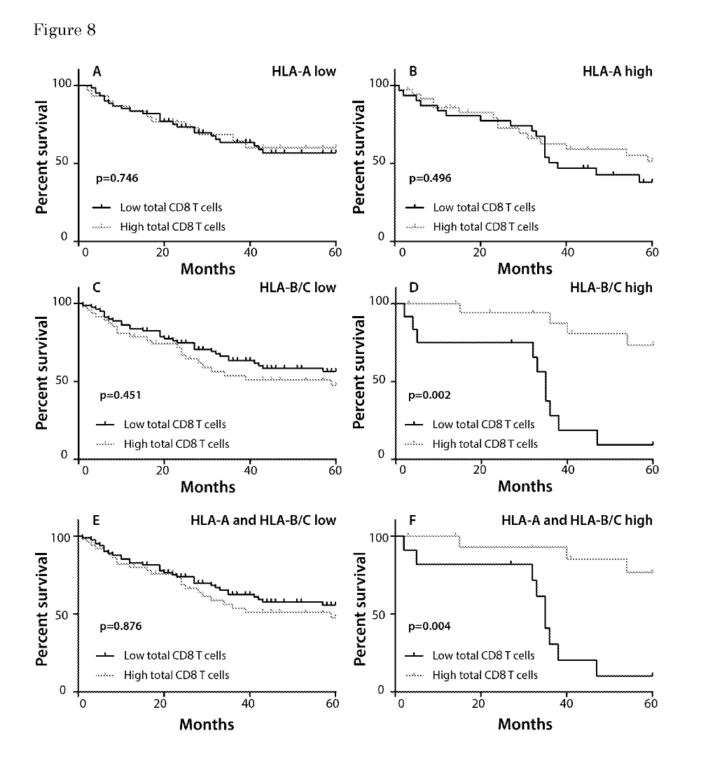

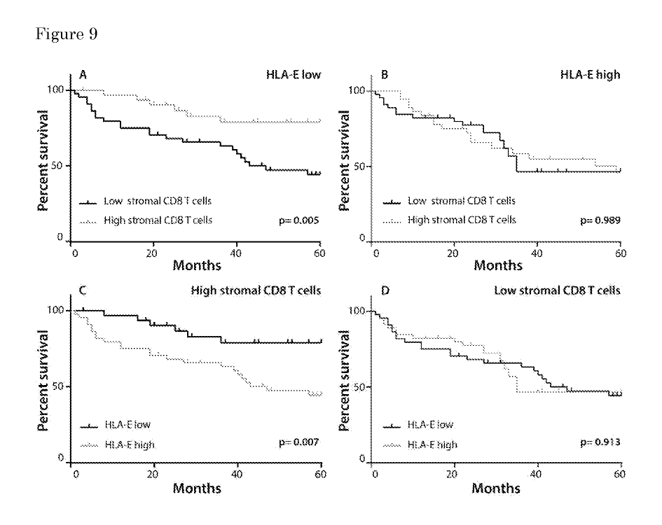

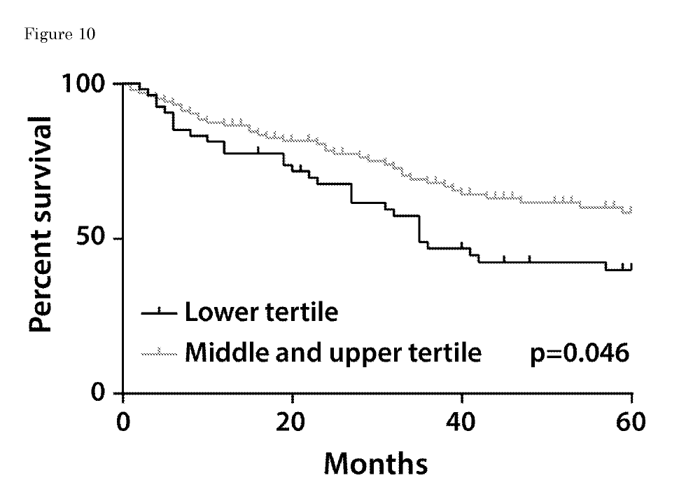

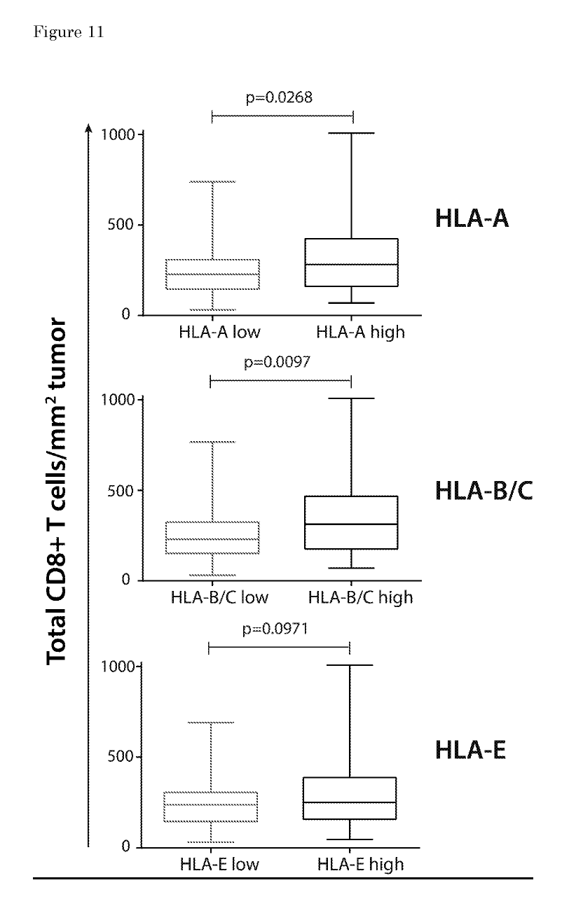

Subsequently, we analyzed the distribution of the inhibitory receptors, including NKG2A, on tumor infiltrating lymphocytes. A flow cytometry panel of 9 antibodies and a live/dead marker was designed to determine frequencies of CD8 T cell subsets expressing combinatorial profiles of co-inhibitory receptors in 14-21 day TIL cultures of oropharyngeal carcinomas. Expression of the inhibitory receptor NKG2A ranged from 5-60% (average 25%) of intratumoral CD8 T cells, whereas blood frequencies rarely exceed 5% (FIG. 2A). All these lymphocytes co-expressed the partner CD94 to result in functional receptors. These frequencies were quite comparable to those found in our earlier studies in cervical carcinoma (Gooden M J M, Lampen M, Jordanova E S, Leffers N, Trimbos J B, van der Burg S H, Nijman H, van Hall T: HLA-E expression by gynecological cancers restrains tumor-infiltrating CD8⁺ T lymphocytes. In: Proc Natl Acad Sci USA. vol. 108; 2011: 10656-10661). Multicolor flow cytometry analysis revealed that within the NKG2A+CD8 T cell populations approximately 35% did not express the inhibitory receptors CTL-A4, PD-1 or TIM3 (FIGS. 2B and C), suggesting that these cells can only be targeted by checkpoint blockade of NKG2A and not to the known other immune checkpoints tested for. Of course, combination of checkpoint blockers have been demonstrated to mediate superior clinical effects, most likely due to compensatory mechanisms (Curran M A, Montalvo W, Yagita H, Allison J P: PD-1 and CTLA-4 combination blockade expands infiltrating T cells and reduces regulatory T and myeloid cells within B16 melanoma tumors. In: Proc Natl Acad Sci USA. vol. 107; 2010: 4275-4280. Wolchok J D, Kluger H, Callahan M K, Postow M A, Rizvi N A, Lesokhin A M, Segal N H, Ariyan C E, Gordon R-A, Reed K et al: Nivolumab plus ipilimumab in advanced melanoma. In: N Engl J Med. vol. 369; 2013: 122-133). Therefore our preliminary data analyses on TIL subsets qualifies the NKG2A-HLA-E axis as a major negative regulator of anti-tumor immunity and is a basis for development of NKG2A blocking antibodies for the oncology clinic.

HLA-E and NKG2A⁺ T Cells are Strongly Increased after Immunotherapy in Different Mouse Tumor Models.

Clinical applications of immunotherapy in our department is geared towards the HPV-induced cancers cervical carcinoma and oropharyngeal carcinoma, and metastatic melanoma. Mouse models for HPV-induced cancer (TC-1) and melanoma (B16F10) have been instrumental in the development of these clinical initiatives. In both mouse models we now investigated the role for CD94/NKG2A in T cell immunity and therapy-induced tumor control. Established B16F10 melanomas were treated with adoptive transfer of TCR-transgenic pmel T cells, which were subsequently activated in vivo by peptide vaccination (FIG. 3A, B). This protocol results in complete tumor control in some of the animals and a clear delay in tumor outgrowth in the other animals. Whereas the expression of Qa-1 (the mouse HLA-E homolog) on in vitro cultured B16F10 cells and B16F10 cells from in vivo growing tumors is hardly detectable (FIG. 3C), tumor cells from mice that had been treated with immunotherapy displayed clearly enhanced levels of Qa-1. This indicated that immune activation results in upregulation of Qa-1, quite similar as found for PD-L1. The increase of such inhibitory ligands is most likely mediated by IFNg as a means of negative feedback to protect the tissues for immunopathology. In the very same tumors we analyzed the expression of NKG2A and CD94 on infiltrating CTL. Untreated control tumors contained between 10-20% NKG2A⁺ CD8 T cells (FIG. 3D), a percentage that is within the range as found in human cancers. Immunotherapy, however, strongly increased this frequency up to 65%. The frequency of NKG2A⁺ NK cells did not alter by immunotherapy, but were already above 50%. Of note, these stainings were performed with the well-known ‘20d5’ antibody detecting also other family members of the NKG2 family, but were confirmed with the more NKG2A-specific antibody 16A11.

Very comparable data were obtained in the HPV-induced TC1 tumor model in which vaccination with synthetic long peptide is applied as form of immunotherapy (FIG. 4). Levels of Qa-1 on the surface of TC1 tumor cells were clearly increased by immunotherapy and frequencies of NKG2A⁺ T cells were strongly increased also in this model (FIG. 4A-F). Therapeutic vaccination not only increased the number of tumor-infiltrating CD8 T cells but also resulted in the expression of NKG2A on the large majority of tumor-infiltrating CD8 T cells (FIG. 4F), indicating that local immune activation and release of pro-inflammatory cytokines triggers suppressive feedback mechanisms, among which NKG2A. In the TC1 tumor model we furthermore observed a preference of tumor-specific CD8 T cells to induce NKG2A compared to bystander activated CD8 T cells and, finally, that therapeutic vaccination actively recruited high numbers of NKG2A⁺ NK cells to the tumor site (FIG. 4G-H).

These data show that B16F10 and TC-1 tumor models are excellently suited to study the immunotherapeutic potential of NKG2A-blockade as a single agent or in combination with several other forms of (immune)therapy. Together these data from mouse models firmly underscores the great therapeutic potential of blocking antibodies to NKG2A, especially in combination with strong vaccines, to unleash the cytotoxic force of NK and CD8 T cells.

### Blocking NKG2A Receptor Increases CTL Function In Vitro

As a first indication if blocking the inhibitory receptor NKG2A would indeed releases the break from CD8 T cell activation, we selected mouse and human CTL clones with known specificity. These T cells were in vitro incubated with peptide-loaded target cells for TCR-mediated activation in the presence or absence of blocking antibodies to NKG2A (20d5 for mouse and 2199 for human). Blockade of NKG2A with antibody 20D5 increased mouse CTL reactivity in a dose-dependent manner (FIG. 5A-B). The highest concentration of blocking antibody resulted in tripled release of IFNg. Similarly, incubation of the human CTL clone with cognate peptide and a blocking antibody to NKG2A led to increased reactivity. Interestingly, the human CTL clone did not homogeneously express CD94/NKG2A and measurement of T cell activation at the single cell level with flow cytometry showed that only the reactivity of CTL displaying the inhibitory receptor could be augmented when NKG2A was blocked. The NKG2A-negative T cell subset within this culture were not affect in this system, demonstrating on-target specificity of the antibody (FIG. 5C). Thus, these data suggest that NKG2A⁺ CTL have a superior activation potential compared to NKG2A− CTL.

### Example 2

To investigate the prognostic value of CD8⁺ tumor infiltrating T cells in the context of HLA-A, B and C as well as HLA-E and its association with overall survival (OS), we retrospectively studied a group of 197 patients with non-small cell lung cancer (NSCLC). We focused on pulmonary adenocarcinoma not only because this is the main histological subtype in NSCLC (Herbst 2008, Alberg 2005) but also because HLA loss has been reported to be less frequent than in squamous cell carcinoma, the other major subtype of NSCLC (Baba 2013, Hanagiri 2013a, Hanagiri 2013b Kikuchi 2007, Korkolopoulou 1996) and therefore is expected to benefit the most from active T-cell-mediated immunotherapy. Our data revealed that the expression of HLA-E by tumor cells was an independent prognostic factor for OS. High expression of HLA-E neutralized the positive prognostic value of high stromal CD8⁺ T cell infiltration in NSCLC.

### Materials and Methods

### Study Population

We retrospectively identified 197 patients diagnosed with non-small cell lung cancer (NSCLC), subtype adenocarcinoma, in the Leiden University Medical Center (LUMC) between 2000 and 2013. All patients underwent preoperative staging and were classified as stage I/II NSCLC and subsequently underwent surgical resection of the primary tumor with systematic lymph node dissection. After surgical removal of the tumor and its draining lymph nodes, patients were considered disease free. Tumor tissue, clinical data and follow-up data were collected from all patients. Staging of NSCLC was determined according to the TNM (Tumor, Node, Metastasis) classification using the updated guidelines of the International Association for the Study of Lung Cancer (IASLC)(Tanoue 2009). The use of archival tumor blocks was in accordance with guidelines from the Dutch Federation of Medical Research Association. Since this retrospective study does not fall under the scope of the Medical Research Involving Human Subjects Act (WMO), it was not subject to a prior review by a Medical Ethical Committee and written informed consent was not obtained. However, patient data were anonymized.

### Antibodies

Mouse monoclonal antibodies HCA-2 (anti HLA-A, 1:1000) and HC-10 (anti HLA B/C, 1:500) were used to detect expression of the free heavy chain of the HLA class I molecule. Rabbit anti-human β2-microglobulin (anti-β2M; clone A-072, DAKO, 1:2000) and mouse anti-human HLA-E (clone MEM-E/02; Serotec, Germany [1:200]) antibodies were used in order to detect the light chain and non-classical HLA-E heavy chain respectively. Mouse monoclonal CD8 antibody (clone IA5, Leica Biosystems, Germany [1:500]) was used for the detection of the CD8⁺ T-cells.

### Immunochemistry

Formalin-fixed, paraffin embedded tumor blocks were cut in 4 μm sections using a microtome and deparaffinized in xylene. The endogenous peroxidase activity was blocked for 20 minutes using 0.3% hydrogen peroxide/methanol. The samples were subsequently rehydrated in 70% and 50% ethanol and antigen retrieval was performed by heating the samples to 97° C. for 10 minutes in citrate buffer (either pH 9.0 or pH 6.0, DAKO, Glostrup, Denmark). Antibodies were diluted in phosphate buffered saline (PBS, Fresenius Kabi Bad Homburg, Germany) with 1% bovine serum albumin (BSA) and incubated overnight at room temperature. The slides were stained immunohistochemically with horseradish peroxidase (HRP)-conjugated anti-mouse IgG (DAKO envision) for 30 minutes at room temperature. NovaRed (Vector, Burlingame, USA) was applied as a chromagen followed by counterstaining with Mayer's hematoxylin (Klinipath). All washing steps were done with PBS. All slides were mounted with Pertex mounting medium (HistoLab, Sweden).

The microscopic evaluation and analysis of the HCA2, HC10, β2M and HLA-E staining was performed by two independent observers without prior knowledge of clinical or histopathological parameters (observer one 100% of the cohort, observer two 20% of the cohort). The inter-observer agreement was assessed by calculating Cohen's kappa coefficient resulting in a coefficient of >0.70 for all stainings which indicates a substantial inter-observer agreement.

The grade of tumor differentiation was determined and classified as either poorly differentiated, moderately differentiated or well differentiated based on the immunohistochemically stained slides. Expression patterns of the previously mentioned antibodies were assessed according to the scoring system proposed by the Ruiter et a (Ruiter 1998). Using this method the entire slide is screened and the percentage of positive tumor cells was classified as: absent 0%, sporadic 1-5%, local 6-25%, occasional 25-50%, majority 51-75% and large majority 76-100% (1-6). Furthermore, this score includes intensity of the staining which is classified as negative, low, medium and high (0-3). The intensity was noted for all antibodies with the exception of CD8 since high intensity was always observed. The final score was based on both intensity and percentage and was categorized as 1-4 (low expression) and 5-9 (high expression).

### Quantification of Infiltrating CD8⁺ T-Cells

CD8⁺ T-cell infiltration was assessed by screening five randomly captured high resolution (200×) images of each slide. The area of the tumor nests and stromal areas were marked and calculated using NIH-ImageJ software (v1.48). CD8⁺ T cells were counted by area and represented as the number of cells per mm2 of tumor area with a distinction between intraepithelial and stromal CD8⁺ T cells. The mean number of intraepithelial, stromal and total number of tumor-infiltrating CD8⁺ T cells were calculated and patients were dichotomized for high or low CD8⁺ T cell infiltration based on the mean CD8⁺ T cell infiltration for all patients.

### Statistical Analysis

Nonparametric Mann-Whitney U test was used to compare continuous variables between patient groups and group comparisons of categorical data were performed by two-tailed _{X}2 test. Overall survival (OS) was defined as date of surgery until date of death due to any cause, or date of last follow-up with a maximum follow-up time of 5 years. When assessing survival based on HLA expression, low and high expression of HLA indicates the presence of a functional HLA molecule, i.e high expression of both β2M and the HLA heavy chain of HLA-A, HLA-B/C and HLA-E respectively. Survival was estimated by using Kaplan-Meier methodology and the log-rank test was used to compare the two curves. Univariate Cox proportional hazards model was used to study the effect of single determinants on OS. Multivariate Cox regression analysis was performed with variables that reached statistical significance in univariate analysis. Stepwise regression was employed to estimate the final model. Two-sided P values of <0.05 were considered statistically significant. Bonferroni correction was applied for multiple testing. Statistical software package SPSS 20.0 (SPSS, Chicago, Ill.) was used for data analysis. GraphPad Prism 6.02 (Graphpad Software, LA Jolla, Calif.) was used to estimate survival curves.

### Results & Discussion

Stromal CD8 T-Cell Infiltration Correlates Best with Overall Survival.

A cohort of 197 patients with pulmonary adenocarcinoma was evaluated. The grade of differentiation by the tumor was classified as either poor (50%), moderate (33%) or well differentiated (17%). In 31% of cases, patients had advanced disease (stage III/IV) despite being classified as stage I/II based on pre-operative diagnostic modalities (Table 1). Mean age was 66 years (range 37-90 years) and the number of males (n=99) and females (n=98) was evenly distributed.

The extent of CD8⁺ T-cell infiltration was studied by enumeration of intraepithelial and stromal CD8⁺ T cells in tumor sections. Examples of representative immunohistochemical stainings of CD8⁺ T cells are displayed in FIG. 6. Overall intraepithelial CD8⁺ T-cell infiltration ranged from 7 to 1460 cells/mm2 tumor (mean 194; median 150), stromal CD8⁺ T cells from 35 to 1332 cells/mm2 tumor (mean 348; median 320) and total CD8⁺ T cells from 32 to 1008 cells/mm2 tumor (mean 271; median 246). There were no differences in total CD8⁺ T-cell tumor infiltration between males and females (chi square test, p=0.267). Patients were divided in two groups with low or high CD8⁺ T cell infiltration, based on the mean CD8⁺ T-cell count for all patients, and the association with OS was plotted. A relatively strong stromal CD8⁺ T-cell infiltration displayed the best association with a beneficial clinical outcome (log-rank test, p=0.068; FIG. 7 A-C). The negative effect of low stromal CD8⁺ T-cell infiltration was magnified when the patients were divided on the basis of tertiles, with patients in the lower tertile defined as having low CD8⁺ stromal T cell infiltration and the other patients as having high stromal CD8⁺ T cell infiltration (p=0.046, FIG. 10), similar to what was reported before (Al-Shibli 2008, Bremnes 2011, Djenidi 2015, Donnem 2015, Hiraoka 2006).

### Interaction Between Classical HLA Class I Expression and CD8⁺ T Cells.

It can be of interest to identify the factors governing a successful attack of NSCLC by CD8⁺ T cells as illustrated by the facts that a) more than 40% of NSCLC patients respond to checkpoint inhibitor therapy (Garon 2015, Gettinger 2015, Jia 2015); and b) especially those patients are likely to respond in whom the tumor has generated neo antigens for CD8⁺ T cells (Rizvi 2015). One of the key molecules in this process is the expression of HLA molecules required to present tumor-specific peptides to T cells. When measured with a pan-HLA class I antibody, the loss of HLA is observed in almost half of the patients with pulmonary adenocarcinoma (Baba 2013, Hanagiri 2013a, Hanagiri 2013b Kikuchi 2007, Kikuchi 2008). We used antibodies to distinct the expression of HLA-A and HLA-B/C in order to chart the HLA loss in more detail Assessment of the expression of classical HLA class I molecules was performed using antibodies against 62-M, HLA-A and HLA-B/C (FIG. 6). 62-M was expressed in 76% of cases, but HLA-A and HLA-B/C were expressed in only 56% and 25% of the cases, respectively (Table 1). Thus, we found that HLA-A was decreased in about 40% of the patients while the decrease in HLA-B/C expression was even as high as 75% which is in line with only one other study that reports specifically on loss of HLA-B/C in NSCLC (Ramnath 2006). Subsequently, the association between tumor stage, HLA class I molecules and CD8⁺ T cell infiltration was assessed (Table 3). High expression of HLA-A strongly correlated with high expression of HLA-B/C (p=0.0001). A clear correlation existed between the presence or absence of functional HLA class I expression and the total number of tumor-infiltrating CD8⁺ T cells. Tumors with downregulation of HLA-A (p=0.012) or HLA-B/C (p=0.018) displayed on average lower numbers of total tumor-infiltrating T cells (Table 3 and FIG. 11).

When patients were grouped according to a low or high expression of HLA-A or HLA-B/C, Kaplan Meier curves did not reveal any direct impact of classical HLA class I expression on clinical outcome (FIGS. 7D and 7E). However, an interaction analysis between classical HLA expression and total CD8⁺ T cell infiltration in tumor tissue revealed a clear beneficial effect of a dense CD8⁺ T cell infiltration in HLA-B/C positive tumors (HR 0.212, 95% CI 0.074-0.606, p=0.004) or HLA-A and HLA-B/C-positive tumors (HR 0.215, 95% CI 0.069-0.673, p=0.008) with respect to OS (Table 2 and FIG. 8). This was not the case when CD8⁺ T-cell infiltration was analyzed in the context of HLA-A expression only. Thus the interaction analyses of HLA expression and CD8⁺ T-cell infiltration led to the novel observation that the prognostic effect of a dense CD8⁺ T-cell tumor infiltration is only retained when tumors display a high expression of classical HLA class I, in particular HLA-B/C (FIG. 8).

### HLA-E Expression is a Strong Negative Determinant for OS.

Other key molecules governing a successful attack of T cells in NSCLC are the so-called checkpoints (Pan 2015). The non-classical HLA-E molecule is the ligand for the inhibition receptor CD94/NKG2A and represents an important immunologic checkpoint (Kochan 2013, van Hall 2010). In more than 70% of pulmonary adenocarcinoma cases a high expression of HLA-E was observed (FIG. 6F and Table 1). The high expression of HLA-E was associated with worse OS (HR 0.632, 95% CI 0.406-0.984, p=0.042; Table 2 and FIG. 7F). This study is the first to show that a high expression of the non-classical HLA-E molecule affects overall survival in NSCLC.

Since both stromal CD8⁺ T-cell infiltration and the expression of HLA-E displayed the strongest effects on overall survival as a single determinant (FIGS. 7B and 7F, FIG. 10), a subsequent analysis was performed to study the interaction between these two factors. Clearly, a dense stromal CD8⁺ T cell infiltration showed a strong positive prognostic value in HLA-E negative tumors (HR 0.303, 95% CI 0.124-0.741, p=0.009; FIGS. 9A and 9B). However, this beneficial effect of a dense stromal CD8⁺ T cell infiltration disappears in patients with high expression of HLA-E (HR 1.004, 95% CI 0.550-1.835, p=0.989; FIGS. 9C and 9D). In conclusion, the beneficial effect displayed by tumor-infiltrating stromal CD8⁺ T cells is impeded when HLA-E is highly expressed by tumors. The expression of HLA-E can inhibit the function of T lymphocytes and natural killer (NK) cells when it engages with CD94/NKG2A (Kochan 2013, van Hall 2010, Ulbrecht 1999), as well as activate these cells when HLA-E engages with CD94/NKG2C (Guma 2005). A few studies in breast cancer and cervical adenocarcinoma have reported survival benefit for HLA-E expressing tumors (de Kruijf 2010, Spaans 2012) while others, similar to us, reported a negative effect of HLA-E on OS in ovarian cancer, colorectal cancer and gastric cancer (Gooden 2011, Bossard 2012, Ishigami 2015, Zhen 2013). Potentially, the type of receptor for HLA-E expressed by CD8 T cells is at the basis of this difference. In ovarian cancer and colorectal cancer the T cells were shown to express the inhibitory receptor CD94/NKG2A (Gooden 2011, Bossard 2012). In line with previous studies in NSCLC, a dense stromal CD8⁺ T-cell tumor-infiltrate was associated with longer OS (FIG. 7 and FIG. 10) (Al-Shibli 2008, Bremnes 2011, Djenidi 2015, Donnem 2015, Hiraoka 2006, Schalper 2015). In our study, a high expression of HLA-E by tumor cells clearly had a negative effect on CD8⁺ T cells. The positive prognostic effect of stromal CD8⁺ T cells on OS was only apparent in patients with low expression of HLA-E on their tumor cells. A high tumor expression of HLA-E completely abolished the prognostic effect of CD8⁺ T-cell infiltrate (Table 2 and FIG. 9).

### HLA-E Expression is an Independent Determinant of OS in Pulmonary Adenocarcinoma.

In order to assess the effect of each single variable on the relative risk of death, univariate and multivariate Cox proportional hazards analysis were performed to quantify survival differences (Table 2). Tumor stage and male gender have been reported before as negative risk factors for OS in pulmonary adenocarcinoma[32] and indeed in our cohort high stage tumors (stage I/II vs stage III/IV, HR 0.619, 95% CI 0.399-0.961, p=0.033) as well as male gender (HR 1.834, 95% CI 1.184-2.839, p=0.007) were associated with worse OS. In the univariate analysis, a low expression of non-classical HLA-E by tumor cells was associated with a strong reduced risk of death in this cohort (HR 0.632, 95% CI 0.406-0.984, p=0.042). Presence of high stromal CD8⁺ T cells correlated with improved OS and reached near-significance (HR 1.560, 95% CI 0.962-2.530, p=0.072) and hence was included in the multivariate analysis together with tumor stage, gender and HLA-E expression.

Similar to the univariate analysis the positive effect of stromal CD8⁺ T cells on OS approached statistical significance (HR 1.613, 95% CI 0.993-2.620, p=0.054) in the multivariate analysis. In addition to tumor stage and gender, the increased expression of HLA-E was significantly associated with OS (HR 0.612, 95% CI 0.392-0.956, p=0.031) indicating that low HLA-E expression is an independent positive prognostic factor for OS in pulmonary adenocarcinoma.

Our results showed that about 70% of the pulmonary adenocarcinomas displayed a high expression of HLA-E (Table 1). In view of its effect on both T cells and NK cells, blocking HLA-E and/or its CD94-NKG2A inhibitory receptor may form a valuable target for the immunotherapy of NSCLC. Treatment with anti-NKG2A monoclonal antibody was shown to overcome HLA-E mediated suppression of anti-tumor cellular cytotoxicity in vitro (Levy 2009, Derre 2006) and this has resulted in a currently ongoing phase I/II trial in which patients with advanced head and neck cancer are treated with an anti-NKG2A monoclonal antibody (ClinicalTrials.gov, Identifier: NCT02331875).

TABLE 1
Table 1 Overview of stage, differentiation and immuno-
histochemical expression patterns in pulmonary adenocarcinoma.
Surgical-pathological staging (number, %)

| | |
|---|---|
| I | 62 (31%) |
| II | 74 (38%) |
| III | 35 (18%) |
| IV | 26 (13%) |

Differentiation (number, %)

| | |
|---|---|
| Poor | 98 (50%) |
| Moderate | 66 (33%) |
| Well | 33 (17%) |

β2-M (number, %)

| | |
|---|---|
| Low | 47 (24%) |
| High | 150 (76%) |

HLA-A (number, %)

| | |
|---|---|
| Low | 87 (44%) |
| High | 110 (56%) |

HLA-B/C (number, %)

| | |
|---|---|
| Low | 148 (75%) |
| High | 49 (25%) |

HLA-E (number, %)

| | |
|---|---|
| Low | 55 (28%) |
| High | 142 (72%) |

Total CD8+ (number, %)

| | |
|---|---|
| Low | 96 (59%) |
| High | 68 (41%) |

CD8+ in tumor (number, %)

| | |
|---|---|
| Low | 104 (64%) |
| High | 59 (36%) |

CD8+ in stroma (number, %)

| | |
|---|---|
| Low | 92 (56%) |
| High | 71 (44%) |

TABLE 2
Table 2. Univariate and multivariate Cox proportional hazard analysis.

| | | Univariate analysis | | Multivariate analysis | |
|---|---|---|---|---|---|
| Variable | | HR (95% CI) | p value | HR (95% CI) | p value |
| Stage | I/II vs III/IV | 0.619 (0.399-0.961) | 0.033 | 0.587 (0.377-0.913) | 0.018 |
| Sex | Male vs Female | 1.834 (1.184-2.839) | 0.007 | 1.785 (1.152-2.765) | 0.009 |
| Differentiation | poor vs medium/well | 1.423 (0.928-2.182) | 0.106 | | |
| β2-microglobulin | low vs high | 0.762 (0.442-1.314) | 0.328 | | |
| HLA-A | low vs high | 0.703 (0.462-1.084) | 0.112 | | |
| HLA-B/C | low vs high | 0.822 (0.498-1.358) | 0.443 | | |
| HLA-E | low vs high | 0.632 (0.406-0.984) | 0.042 | 0.612 (0.392-0.956) | 0.031 |
| Intraepithelial CD8 | low vs high | 0.682 (0.427-1.087) | 0.108 | | |
| Stromal CD8 | low vs high | 1.560 (0.962-2.530) | 0.072 | 1.613 (0.993-2.620) | 0.054 |
| Total CD8 | low vs high | 1.130 (0.705-1.812) | 0.659 | | |
| HLA-E low | high vs low stromal CD8 | 0.303 (0.124-0.741) | 0.009 | | |
| HLA-E high | high vs low stromal CD8 | 1.004 (0.550-1.835) | 0.989 | | |
| Stromal CD8 high | high vs low HLA-E | 3.282 (1.308-8.232) | 0.011 | | |
| Stromal CD8 low | high vs low HLA-E | 1.032 (0.585-1.818) | 0.914 | | |
| HLA-B/C high | high vs low total CD8 | 0.212 (0.074-0.606) | 0.004 | | |
| HLA-A and B/C high | high vs low total CD8 | 0.215 (0.069-0.673) | 0.008 | | |

Significant differences (p < 0.05) are indicated in bold

TABLE 3
Table 3: Relationship of tumor characteristics with HLA expression and CD8+ T cell
expression in pulmonary adenocarcinoma.

| | HLA-A | | | HLA-B/C | | | HLA-E | | |
|---|---|---|---|---|---|---|---|---|---|
| | High | Low | P value | High | Low | P value | High | Low | P value |
| Stage | | | | | | | | | |
| I | 36 | 26 | 0.621 | 17 | 45 | 0.872 | 44 | 18 | 0.777 |
| II | 43 | 31 | | 16 | 58 | | 56 | 18 | |
| III | 16 | 19 | | 9 | 26 | | 25 | 10 | |
| IV | 15 | 11 | | 7 | 19 | | 17 | 9 | |
| β2-M | | | | | | | | | |
| Low | 18 | 29 | 0.007 | 8 | 39 | 0.179 | 32 | 15 | 0.576 |
| High | 92 | 58 | | 41 | 109 | | 110 | 40 | |
| HLA-A | | | | | | | | | |
| Low | | | | 6 | 81 | 0.0001 | 60 | 27 | 0.426 |
| High | | | | 43 | 67 | | 82 | 28 | |
| HLA-B/C | | | | | | | | | |
| Low | | | | | | | 106 | 42 | 0.856 |
| High | | | | | | | 36 | 13 | |
| Total CD8+ | | | | | | | | | |
| Low | 41 | 55 | 0.012* | 16* | 80 | 0.018* | 64 | 32 | 0.480* |
| High | 45 | 23 | | 25 | 43 | | 53 | 15 | |
| CD8+ in stroma | | | | | | | | | |
| Low | 45 | 47 | 0.819* | 19 | 73 | 0.444* | 66 | 26 | 0.990* |
| High | 41 | 30 | | 22 | 49 | | 51 | 20 | |
| CD8+ in tumor | | | | | | | | | |
| Low | 50 | 54 | 0.426* | 21 | 83 | 0.186* | 68 | 36 | 0.057* |
| High | 36 | 23 | | 20 | 39 | | 49 | 10 | |

Significant results (p < 0.050) are indicated in bold.
*Bonferroni corrected p value Citations in example 2

Alberg A J, Brock M V, Samet J M. Epidemiology of lung cancer: looking to the future. J Clin Oncol 2005; 23:3175-85.
Al-Shibli K I, Donnem T, Al-Saad S, Persson M, Bremnes R M, Busund L T. Prognostic effect of epithelial and stromal lymphocyte infiltration in non-small cell lung cancer. Clin Cancer Res 2008; 14:5220-7.
Baba T, Shiota H, Kuroda K, Shigematsu Y, Ichiki Y, Uramoto H et al. Clinical significance of human leukocyte antigen loss and melanoma-associated antigen 4 expression in smokers of non-small cell lung cancer patients. Int J Clin Oncol 2013; 18:997-1004.
Bossard C, Bezieau S, Matysiak-Budnik T, Volteau C, Laboisse C L, Jotereau F et al. HLA-E/beta2 microglobulin overexpression in colorectal cancer is associated with recruitment of inhibitory immune cells and tumor progression. Int J Cancer 2012; 131:855-63.
Bremnes R M, Al-Shibli K, Donnem T, Sirera R, Al-Saad S, Andersen S et al. The role of tumor-infiltrating immune cells and chronic inflammation at the tumor site on cancer development, progression, and prognosis: emphasis on non-small cell lung cancer. J Thorac Oncol 2011; 6:824-33.
Carosella E D, HoWangYin K Y, Favier B, LeMaoult J. HLA-G-dependent suppressor cells: Diverse by nature, function, and significance. Hum Immunol 2008; 69:700-7.
Derre L, Corvaisier M, Charreau B, Moreau A, Godefroy E, Moreau-Aubry A et al. Expression and release of HLA-E by melanoma cells and melanocytes: potential impact on the response of cytotoxic effector cells. J Immunol 2006; 177:3100-7.
de Kruijf E M, Sajet A, van Nes J G, Natanov R, Putter H, Smit V T et al. HLA-E and HLA-G expression in classical HLA class I-negative tumors is of prognostic value for clinical outcome of early breast cancer patients. J Immunol 2010; 185:7452-9.
Djenidi F, Adam J, Goubar A, Durgeau A, Meurice G, de M, V et al. CD8+CD103+ tumor-infiltrating lymphocytes are tumor-specific tissue-resident memory T cells and a prognostic factor for survival in lung cancer patients. J Immunol 2015; 194:3475-86.
Donnem T, Hald S M, Paulsen E E, Richardsen E, Al-Saad S, Kilvaer T K et al. Stromal CD8+ T-cell Density-A Promising Supplement to TNM Staging in Non-Small Cell Lung Cancer. Clin Cancer Res 2015.
Garon E B, Rizvi N A, Hui R, Leighl N, Balmanoukian A S, Eder J P et al. Pembrolizumab for the treatment of non-small-cell lung cancer. N Engl J Med 2015; 372:2018-28.
Gettinger S N, Horn L, Gandhi L, Spigel D R, Antonia S J, Rizvi N A et al. Overall Survival and Long-Term Safety of Nivolumab (Anti-Programmed Death 1 Antibody, BMS-936558, ONO-4538) in Patients With Previously Treated Advanced Non-Small-Cell Lung Cancer. J Clin Oncol 2015; 33:2004-12.
Gooden M, Lampen M, Jordanova E S, Leffers N, Trimbos J B, van der Burg S H et al. HLA-E expression by gynecological cancers restrains tumor-infiltrating CD8(+) T lymphocytes. Proc Natl Acad Sci USA 2011; 108:10656-61.
Guma M, Busch L K, Salazar-Fontana L I, Bellosillo B, Morte C, Garcia P et al. The CD94/NKG2C killer lectin-like receptor constitutes an alternative activation pathway for a subset of CD8+ T cells. Eur J Immunol 2005; 35:2071-80.
Hanagiri T, Shigematsu Y, Shinohara S, Takenaka M, Oka S, Chikaishi Y et al. Clinical significance of expression of cancer/testis antigen and down-regulation of HLA class-I in patients with stage I non-small cell lung cancer. Anticancer Res 2013a; 33:2123-8.
Hanagiri T, Shigematsu Y, Kuroda K, Baba T, Shiota H, Ichiki Y et al. Prognostic implications of human leukocyte antigen class I expression in patients who underwent surgical resection for non-small-cell lung cancer. J Surg Res 2013b; 181:e57-e63.
Herbst R S, Heymach J V, Lippman S M. Lung cancer. N Engl J Med 2008; 359:1367-80.
Hiraoka K, Miyamoto M, Cho Y, Suzuoki M, Oshikiri T, Nakakubo Y et al. Concurrent infiltration by CD8+ T cells and CD4+ T cells is a favourable prognostic factor in non-small-cell lung carcinoma. Br J Cancer 2006; 94:275-80.
Ishigami S, Arigami T, Okumura H, Uchikado Y, Kita Y, Kurahara H et al. Human Leukocyte Antigen (HLA)-E and HLA-F Expression in Gastric Cancer. Anticancer Res 2015; 35:2279-85.
Jia M, Feng W, Kang S, Zhang Y, Shen J, He J et al. Evaluation of the efficacy and safety of anti-PD-1 and anti-PD-L1 antibody in the treatment of non-small cell lung cancer (NSCLC): a meta-analysis. J Thorac Dis 2015; 7:455-61.
Johnson D B, Rioth M J, Horn L. Immune checkpoint inhibitors in NSCLC. Curr Treat Options Oncol 2014; 15:658-69.
Kikuchi E, Yamazaki K, Torigoe T, Cho Y, Miyamoto M, Oizumi S et al. HLA class I antigen expression is associated with a favorable prognosis in early stage non-small cell lung cancer. Cancer Sci 2007; 98:1424-30.
Kikuchi E, Yamazaki K, Nakayama E, Sato S, Uenaka A, Yamada N et al. Prolonged survival of patients with lung adenocarcinoma expressing XAGE-1b and HLA class I antigens. Cancer Immun 2008; 8:13.
Kochan G, Escors D, Breckpot K, Guerrero-Setas D. Role of non-classical MHC class I molecules in cancer immunosuppression. Oncoimmunology 2013; 2:e26491.
Korkolopoulou P, Kaklamanis L, Pezzella F, Harris A L, Gatter K C. Loss of antigen-presenting molecules (MHC class I and TAP-1) in lung cancer. Br J Cancer 1996; 73:148-53.
Lepin E J, Bastin J M, Allan D S, Roncador G, Braud V M, Mason D Y et al. Functional characterization of HLA-F and binding of HLA-F tetramers to ILT2 and ILT4 receptors. Eur J Immunol 2000; 30:3552-61.
Levy E M, Sycz G, Arriaga J M, Barrio M M, Von Euw E M, Morales S B et al. Cetuximab-mediated cellular cytotoxicity is inhibited by HLA-E membrane expression in colon cancer cells. Innate Immun 2009; 15:91-100.
Lin A, Zhang X, Ruan Y Y, Wang Q, Zhou W J, Yan W H. HLA-F expression is a prognostic factor in patients with non-small-cell lung cancer. Lung Cancer 2011; 74:504-9.
Melief C J, van der Burg S H. Immunotherapy of established (pre)malignant disease by synthetic long peptide vaccines. Nat Rev Cancer 2008; 8:351-60.
Pan Z K, Ye F, Wu X, An H X, Wu J X. Clinicopathological and prognostic significance of programmed cell death ligandl (PD-L1) expression in patients with non-small cell lung cancer: a meta-analysis. J Thorac Dis 2015; 7:462-70.
Schalper K A, Brown J, Carvajal-Hausdorf D, McLaughlin J, Velcheti V, Syrigos K N et al. Objective measurement and clinical significance of TILs in non-small cell lung cancer. J Natl Cancer Inst 2015; 107.
Sakurai H, Asamura H, Goya T, Eguchi K, Nakanishi Y, Sawabata N et al. Survival differences by gender for resected non-small cell lung cancer: a retrospective analysis of 12,509 cases in a Japanese Lung Cancer Registry study. J Thorac Oncol 2010; 5:1594-601.
Siegel R, DeSantis C, Virgo K, Stein K, Mariotto A, Smith T et al. Cancer treatment and survivorship statistics, 2012. CA Cancer J Clin 2012; 62:220-41.
Spaans V M, Peters A A, Fleuren G J, Jordanova E S. HLA-E expression in cervical adenocarcinomas: association with improved long-term survival. J Transl Med 2012; 10:184.
Tanoue L T, Detterbeck F C. New TNM classification for non-small-cell lung cancer. Expert Rev Anticancer Ther 2009; 9:413-23.
Torre L A, Bray F, Siegel R L, Ferlay J, Lortet-Tieulent J, Jemal A. Global cancer statistics, 2012. C A Cancer J Clin 2015; 65:87-108.
Ramnath N, Tan D, Li Q, Hylander B L, Bogner P, Ryes L et al. Is downregulation of MHC class I antigen expression in human non-small cell lung cancer associated with prolonged survival? Cancer Immunol Immunother 2006; 55:891-9.
Rizvi N A, Hellmann M D, Snyder A, Kvistborg P, Makarov V, Havel J J et al. Cancer immunology. Mutational landscape determines sensitivity to PD-1 blockade in non-small cell lung cancer. Science 2015; 348:124-8.
Ruiter D J, Ferrier C M, van Muijen G N, Henzen-Logmans S C, Kennedy S, Kramer M D et al. Quality control of immunohistochemical evaluation of tumour-associated plasminogen activators and related components. European BIOMED-1 Concerted Action on Clinical Relevance of Proteases in Tumour Invasion and Metastasis. Eur J Cancer 1998; 34:1334-40.
Ulbrecht M, Couturier A, Martinozzi S, Pla M, Srivastava R, Peterson P A et al. Cell surface expression of HLA-E: interaction with human beta2-microglobulin and allelic differences. Eur J Immunol 1999; 29:537-47.
van der Drift M A, Karim-Kos H E, Siesling S, Groen H J, Wouters M W, Coebergh J W et al. Progress in standard of care therapy and modest survival benefits in the treatment of non-small cell lung cancer patients in the Netherlands in the last 20 years. J Thorac Oncol 2012; 7:291-8.
van Esch E M, Tummers B, Baartmans V, Osse E M, Ter H N, Trietsch M D et al. Alterations in classical and nonclassical HLA expression in recurrent and progressive HPV-induced usual vulvar intraepithelial neoplasia and implications for immunotherapy. Int J Cancer 2014; 135:830-42.
van Hall T., Oliveira C C, Joosten S A, Ottenhoff T H. The other Janus face of Qa-1 and HLA-E: diverse peptide repertoires in times of stress. Microbes Infect 2010; 12:910-8.
Yie S M, Yang H, Ye S R, Li K, Dong D D, Lin X M. Expression of human leucocyte antigen G (HLA-G) is associated with prognosis in non-small cell lung cancer. Lung Cancer 2007; 58:267-74.
Zhen Z J, Ling J Y, Cai Y, Luo W B, He Y J. Impact of HLA-E gene polymorphism on HLA-E expression in tumor cells and prognosis in patients with stage III colorectal cancer. Med Oncol 2013; 30:482.

1-15. (canceled)

16. A method of treating a subject comprising administering a combination of a vaccine and a CD94/NKG2A and/or a CD94/NKG2B binding antibody or a CD94/NKG2A and/or a CD94/NKG2B binding part thereof, said vaccine comprising an immunogen for eliciting an immune response against an antigen or a nucleic acid molecule encoding said immunogen.

17. The method of claim 16, wherein said immunogen is a tumor-antigen.

18. The method of claim 16, wherein said immunogen is a tumor-specific antigen.

19. The method of claim 16, wherein said CD94/NKG2A and/or a CD94/NKG2B binding antibody or a CD94/NKG2A and/or a CD94/NKG2B binding part thereof reduces signaling of CD94/NKG2A and/or CD94/NKG2B when bound to CD94/NKG2A and/or CD94/NKG2B expressing T-cells or natural killer (NK) cells.

20. The method of claim 16, wherein said CD94/NKG2A and/or a CD94/NKG2B binding antibody or a CD94/NKG2A and/or a CD94/NKG2B binding part thereof blocks binding of the CD94/NKG2A and/or CD94/NKG2B ligand HLA-E to CD94/NKG2A and/or CD94/NKG2B expressing T-cells or natural killer (NK) cells.

21. The method of claim 16, wherein said CD94/NKG2A and/or CD94/NKG2B antibody or a CD94/NKG2A and/or a CD94/NKG2B binding part thereof is a human or humanized antibody or a CD94/NKG2A and/or a CD94/NKG2B binding part thereof, preferably an antibody of subclass IgG4.

22. The method of claim 16, wherein the combination further comprises at least one antibody selected from a CLTA4-binding antibody, a PD-1 binding antibody; a PD-L1 binding antibody; a LAG-3 binding antibody; a VISTA antibody and a TIM3 binding antibody or selected from a CTLA4 binding, a PD-L1 binding, a LAG-3 binding, a VISTA binding, or a TIM3 binding part of said antibody.

23. The method of claim 16, wherein the subject is a cancer patient.

24. The method of claim 23, wherein said subject cancer is a cancer, preferably a solid cancer selected from ovarian carcinoma, head & neck carcinoma, melanoma, cervical carcinoma, pancreatic carcinoma, renal cell carcinoma, lung carcinoma, prostate carcinoma, virus induced carcinoma and colorectal carcinoma.

25. The method of claim 16, wherein the subject is further provided with an immune cell transplant.

26. A pharmaceutical composition comprising vaccine and a CD94/NKG2A and/or CD94/NKG2B binding antibody or a CD94/NKG2A and/or a CD94/NKG2B binding part thereof, wherein said vaccine comprises an immunogen for eliciting an immune response against an antigen or a nucleic acid molecule encoding said immunogen.

27. The pharmaceutical composition according to claim 26, wherein said immunogen is a tumor-antigen.

28. A kit of parts comprising a vaccine composition and a composition comprising a CD94/NKG2A and/or CD94/NKG2B binding antibody or a CD94/NKG2A and/or a CD94/NKG2B binding part thereof, wherein said vaccine comprises an immunogen for eliciting an immune response against an antigen or a nucleic acid molecule encoding said immunogen.

29. A method for preparing an immune cell containing cell product comprising culturing a collection of cells comprising T-cells and/or NK-cells in the presence of an immunogen and a CD94/NKG2A and/or a CD94/NKG2B antibody or a CD94/NKG2A and/or a CD94/NKG2B binding part thereof.

30. The method of claim 29, said method further comprising collecting T-cells and/or NK-cells after culturing said collection of cells.

31. A method for stimulating an immune response in a subject comprising administering a vaccine and a CD94/NKG2A and/or CD94/NKG2B binding antibody or a CD94/NKG2A and/or a CD94/NKG2B binding part thereof to the subject in need thereof, wherein said vaccine comprises an immunogen for eliciting an immune response against an antigen or a nucleic acid molecule encoding said immunogen.

32. The method of claim 31, said method further comprising providing said subject with an immune cell transplant comprising T-cells and/or NK-cells in cultured in the presence of said immunogen and a CD94/NKG2A and/or a CD94/NKG2B antibody or a CD94/NKG2A and/or a CD94/NKG2B binding part thereof.
